(19) United States
(12) Patent Application Publication
(10) Pub. No.: US 2023/0255988 A1
(43) Pub. Date: Aug. 17, 2023

(54) METHOD FOR TREATING NEURODEGENERATIVE DISEASES BY ADMINISTERING BENFOTIAMINE OR DERIVATIVE THEREOF

(71) Applicants: CORNELL UNIVERSITY, Ithaca, NY (US); BURKE NEUROLOGICAL INSTITUTE, White Plains, NY (US)

(72) Inventor: Gary GIBSON, Larchmont, NY (US)

(73) Assignees: CORNELL UNIVERSITY, Ithaca, NY (US); BURKE NEUROLOGICAL INSTITUTE, White Plains, NY (US)

(21) Appl. No.: 18/018,417

(22) PCT Filed: Jul. 29, 2021
(86) PCT No.: PCT/US2021/043687

§ 371 Date: Jan. 27, 2023

Related U.S. Application Data
(60) Provisional application No. 63/058,870, filed on Jul. 30, 2020.

(51) Int. Cl. A61K 31/675; A61K 31/505; A61K 31/506; A61P 25/28

(52) U.S. Cl. CPC A61K 31/675; A61K 31/505; A61K 31/506; A61P 25/28

(57) ABSTRACT

A method for treating a subject having or likely to develop a neurodegenerative disease, wherein the subject is administered a pharmaceutically effective amount of a compound within the generic structure (1) wherein R¹ is either —R or —SR, wherein R is a hydrocarbon group containing 1-20 carbon atoms and optionally containing one or more heteroatoms selected from oxygen, nitrogen, and sulfur; and R² is selected from the group consisting of —OR′, —OPO₃²⁻, and —OC(O)R′, wherein R′ is a hydrogen atom or a hydrocarbon group containing 1-6 carbon atoms; and wherein Formula (1) may include pharmaceutically acceptable salts, solvates, and polymorphs thereof, and wherein the subject may be identified as positive, before or during treatment, for at least one marker, including, for example, amyloid plaques, neurofibrillary tangles, decline in brain glucose metabolism, decline in thiamine diphosphate-dependent enzyme activity, and increase in advanced glycation end products.

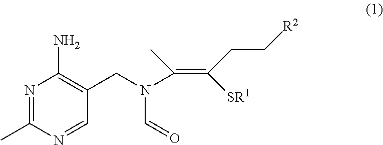

### CROSS REFERENCE TO RELATED APPLICATION

The present application claims benefit of U.S. Provisional Application No. 63/058,870, filed on Jul. 30, 2020, the contents of which are incorporated herein by reference.

### STATEMENT REGARDING FEDERALLY SPONSORED RESEARCH OR DEVELOPMENT

This invention was made with government support under Grant No. R01AG043679 from the U.S. National Institutes of Health (NIH) National Institute on Aging (NIA division). The government has certain rights in the invention.

### FIELD OF THE DISCLOSURE

This disclosure relates generally to methods for treating a neurodegenerative disease, such as Alzheimer's Disease, by administering a pharmaceutically effective amount of a compound in the treatment of the neurodegenerative disease, including by increasing thiamine to pharmacological levels in a subject. The disclosure more specifically relates to such methods in which the compound being administered is a synthetic derivative of thiamine, such as benfotiamine or a derivative thereof.

### BACKGROUND

Numerous methodologies are currently being explored for the treatment of neurodegenerative diseases, such as Alzheimer's Disease (AD) and other cognitive impairments and dementias, including mild cognitive impairment (MCI). Therapies for AD targeting brain amyloid-β (Aβ) have in most cases shown a lack of efficacy. In addition to plaques, tangles, and cognitive decline, multiple changes accompany AD including inflammation, oxidative stress and metabolic dysregulation. Cerebral glucose metabolism, as measured by fluorine-18 (¹⁸F) fluorodeoxyglucose positron-emission tomography (FDGPET), changes decades before AD is typically diagnosed, and in AD patients reductions in glucose utilization correlate highly with cognitive decline (B. A. Gordon et al., The Lancet Neurology, 17, 241-250, 2018; L. Mosconi et al., European Journal of Nuclear Medicine and Molecular Imaging, 36, 811-822, 2009).

Abnormalities in glucose metabolism, vascular changes, and inflammation are known to be closely linked and common features of AD. Thiamine diphosphate (ThDP)-dependent enzymes regulate key steps in brain glucose metabolism, and the activities of ThDP-dependent enzymes decline in blood and brain of AD patients. The reduction in the activity of these enzymes provide a plausible underlying mechanism for the metabolic abnormalities. Thiamine deficiency is known to induce inflammation and change in vasculature. Abnormal metabolism often leads to over-production of free radicals that damage other molecules. At autopsy, oxidative stress in the brain can be as widespread as plaques and tangles (N. Y. Calingasan et al., Journal of Neurochemistry, 72, 751-756, 1999). Increases in advanced glycation end products (AGEs), toxic protein modifications that are indicative of altered glucose metabolism, and their receptor, RAGE, are known to occur in the brain and periphery of AD patients, in both plaques and tangles (Z. S. Atac et al., Journal of Clinical Neuroscience, 59, 197-201, 2019). Moreover, it has been shown that thiamine deficiency increases amyloid plaque production and hyperphosphorylation of tau (Karuppagounder S S, Xu H, Shi Q, Chen L H, Pedrini S, Pechman D, et al. Thiamine deficiency induces oxidative stress and exacerbates the plaque pathology in Alzheimer's mouse model. Neurobiology of Aging. 2009; 30(10):1587-600. doi: 10.1016/j.neurobiolaging.2007.12.013).

As these neurodegenerative diseases arise from an interplay of diverse mechanisms with different levels of expression in different subjects, a more versatile treatment has remained elusive. There would be a benefit in a method that could more generally treat neurodegenerative disease, particularly Alzheimer's Disease, with greater confidence in the outcome.

### SUMMARY OF THE DISCLOSURE

In a first aspect, the present disclosure is directed to a method for treating a subject having or likely to develop a neurodegenerative disease, such as Alzheimer's Disease (AD), by administering to the subject a pharmaceutically effective amount of a compound that is a thiamine derivative, or a lipophilic or non-lipophilic derivative of thiamine. The compound may more particularly be benfotiamine or a derivative thereof.

In some embodiments, the neurodegenerative disease is characterized by mild cognitive impairment (MCI) or more particularly, mild dementia, which may correspond to mild, early, or pre-symptomatic AD. In some embodiments, the neurodegenerative disease is characterized by amyloid plaques and/or neurofibrillary tangles in the brain. The neurodegenerative disease may alternatively or in addition be characterized by decline in brain glucose metabolism, decline in thiamine diphosphate-dependent enzyme activity in the blood or brain, or an increase in advanced glycation end products in the blood, urine, or brain. In some embodiments, the subject is a carrier of apolipoprotein E4 (apoE4), while in other embodiments, the subject is not a carrier of apoE4.

In particular embodiments, the compound being administered has a structure within the following generic structure:

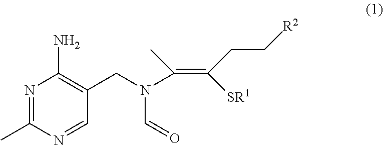

wherein R¹ is either —R or —SR, wherein R is a hydrocarbon group containing 1-20 carbon atoms and optionally containing one or more heteroatoms selected from oxygen, nitrogen, and sulfur; and R² is independently selected from the group consisting of —OR′, —OPO₃²⁻, and —OC(O)R′, or R² is more particularly selected from —OR′ or —OC(O)R′, or R² is specifically —OPO₃²⁻, or specifically —OR′, or specifically —OC(O)R′, wherein R′ is a hydrogen atom or a hydrocarbon group containing 1-6 carbon atoms; and wherein Formula (1) includes pharmaceutically acceptable salts, solvates, and polymorphs thereof. In further embodiments, R¹ is R, wherein R may be an unsaturated hydrocarbon, such as phenyl, or R may be —C(O)R^{a}, wherein R^{a} may be a hydrocarbon group containing 1-20 carbon atoms and optionally containing one or more heteroatoms selected from oxygen, nitrogen, and sulfur. In some embodiments, R^{a} is an unsaturated hydrocarbon or more particularly phenyl. In some embodiments, the compound according to Formula (1) is benfotiamine, or a pharmaceutically acceptable salt, solvate, or polymorph thereof.

In some embodiments, the subject undergoing treatment with a compound of Formula (1) is first tested for the presence of one or more markers (i.e., a biomarker, such as a pathological marker) in the subject before the method of treatment (i.e., before administering the compound) described above, wherein the markers are selected from the group consisting of amyloid plaques in the brain, neurofibrillary tangles in the brain, decline in brain glucose metabolism, decline in thiamine diphosphate-dependent enzyme activity in the blood or brain, and increase in advanced glycation end products in the blood, urine, or brain. In some embodiments, the subject is positive for one or more markers described above before or during the administration of the compound. In some embodiments, the subject is selected on the basis that the subject is positive for the presence of one or more markers described above. In some embodiments, the method further comprises monitoring the subject during the treatment to determine whether a decrease is observed in the level of one or more of any of the markers described above. In some embodiments, the subject is tested for the apolipoprotein E4 (apoE4) gene (allele) before the treatment. In some embodiments, the subject is determined to not be a carrier of apoE4, in which case the subject may be selected for the treatment on the basis that the subject is not a carrier of apoE4. In some embodiments, the neurodegenerative disease is Alzheimer's Disease. In other embodiments, the neurodegenerative disease is mild cognitive impairment. In some embodiments, the mild cognitive impairment is mild dementia.

In some embodiments, the method described above also includes testing the subject for the presence of one or more markers in the subject before the treatment, wherein the markers are selected from amyloid plaques in the brain, neurofibrillary tangles in the brain, decline in brain glucose metabolism, decline in thiamine diphosphate-dependent enzyme activity in the blood or brain, and increase in advanced glycation end products in the blood, urine, or brain.

In other embodiments, the method described above also includes selecting the subject for treatment on the basis that the subject is positive for the presence of one or more markers selected from the group consisting of amyloid plaques in the brain, neurofibrillary tangles in the brain, decline in brain glucose metabolism, decline in thiamine diphosphate-dependent enzyme activity in the blood or brain, and increase in advanced glycation end products in the blood, urine, or brain.

In other embodiments, the method described above also includes monitoring the subject during the treatment to determine whether a decrease is observed in the level of one or more markers selected from the group consisting of amyloid plaques in the brain, neurofibrillary tangles in the brain, decline in brain glucose metabolism, decline in thiamine diphosphate-dependent enzyme activity in the blood or brain, and increase in advanced glycation end products in the blood, urine, or brain.

In other embodiments, in the method described above, the subject is not a carrier of apolipoprotein E4 (apoE4) allele. In other embodiments, in the method described above, the subject is a carrier of apolipoprotein E4 (apoE4) allele. In other embodiments, the method described above further includes testing the subject for the presence of apoE4 allele before the treatment. In some embodiments, in the method described above, the subject has been selected for the treatment on the basis that the subject is not a carrier of apolipoprotein E4 (apoE4).

In some embodiments of the above method (first aspect), the subject may be administered at least 300 mg per day, or at least 600 mg per day, or at least 900 mg per day, or at least 1200 mg per day of any of the compounds described above, which may be a thiamine derivative such as encompassed by Formula (1). In some embodiments, the subject may be administered a daily dosage between 300-1500 mg, 600-1250 mg, 900-1200 mg, 900-1500 mg, or 1200-1500 mg of a compound of Formula (1) per day. In different embodiments, the daily dosage is achieved by two or three administrations. In different embodiments, the daily dosage may be administered for at least six, twelve, or eighteen months.

In a second aspect, the present disclosure is directed to a method for treating a subject, by administering to a subject who has been identified as positive for one or more markers a pharmaceutically effective amount of a compound within the generic structure of Formula (1), wherein the markers are selected from amyloid plaques in the brain, neurofibrillary tangles in the brain, decline in brain glucose metabolism, decline in thiamine diphosphate-dependent enzyme activity in the blood or brain, and increase in advanced glycation end products in the blood, urine, or brain. In some embodiments, the subject does not yet display cognitive impairment at the start of treatment. The method may also include monitoring the level of the at least one marker in the subject. In some embodiments, the administration of the compound is adjusted based on an observed change in the level of the at least one marker. In a further embodiment, the adjustment includes terminating the administration when the level of the at least one marker is diminished. In some embodiments, the subject is a carrier of apolipoprotein E4 (apoE4), while in other embodiments, the subject is not a carrier of apoE4.

In some embodiments of the above method (second aspect), R¹ in Formula (1) is either —R or —SR, wherein R is a hydrocarbon group containing 1-20 carbon atoms and optionally containing one or more heteroatoms selected from oxygen, nitrogen, and sulfur; and R² is independently selected from the group consisting of —OR′, —OPO₃²⁻, and —OC(O)R′, or R² is more particularly selected from —OR′ or —OC(O)R′, or R² is specifically —OPO₃²⁻, or specifically —OR′, or specifically —OC(O)R′, wherein R′ is a hydrogen atom or a hydrocarbon group containing 1-6 carbon atoms; and wherein Formula (1) includes pharmaceutically acceptable salts, solvates, and polymorphs thereof. In further embodiments of the above method, R¹ is R, wherein R may be an unsaturated hydrocarbon, such as phenyl, or R may be —C(O)R^{a}, wherein R^{a} may be a hydrocarbon group containing 1-20 carbon atoms and optionally containing one or more heteroatoms selected from oxygen, nitrogen, and sulfur. In some embodiments of the above method, R^{a} is an unsaturated hydrocarbon or more particularly phenyl. In some embodiments of the above method, the compound according to Formula (1) is benfotiamine, or a pharmaceutically acceptable salt, solvate, or polymorph thereof.

In some embodiments of the above method (second aspect), the subject may be administered at least 300 mg per day, or at least 600 mg per day, or at least 900 mg per day, or at least 1200 mg per day of any of the compounds described above, which may be a thiamine derivative such as encompassed by Formula (1). In some embodiments, the subject may be administered a daily dosage between 300-1500 mg, 600-1250 mg, 900-1200 mg, 900-1500 mg, or 1200-1500 mg of a compound of Formula (1) per day. In different embodiments, the daily dosage is achieved by two or three administrations. In different embodiments, the daily dosage may be administered for at least six, twelve, or eighteen months.

In a third aspect, the present disclosure is directed to a method of increasing a peripheral blood level of thiamine in a subject having a decreased tissue level or peripheral blood level of thiamine, wherein the decreased tissue level or peripheral blood level of thiamine is not due to a dietary insufficiency of thiamine. The method includes administering to the subject a pharmaceutically effective amount of a compound of Formula (1) as described above. In some embodiments, administering to the subject a pharmaceutically effective amount of the compound also increases a tissue level or peripheral blood level of thiamine diphosphate and of thiamine monophosphate in the subject. In some embodiments, the compound is administered daily for a period of at least or more than six, eight, ten, or twelve months, and wherein the tissue level or peripheral blood level of thiamine in the subject at the end of treatment is increased at least or more than 100-fold relative to the peripheral blood level of thiamine prior to treatment. Any of the daily dosages and schedules further described and exemplified throughout this disclosure may be used in the above method to increase a tissue level or peripheral blood level of thiamine. In some embodiments, the subject has a decreased tissue level of thiamine. The subject having a decreased tissue level of thiamine may or may not display cognitive deficits associated with a neurodegenerative condition, such as dementia, mild cognitive impairment, or Alzheimer's disease. In embodiments, the subject has a decreased blood or tissue thiamine level compared to a reference level of tissue thiamine in a subject of the same species having a normal blood or tissue thiamine level, wherein the subject having a normal level of tissue thiamine may or may not suffer from a neurodegenerative condition, such as dementia, mild cognitive impairment, or Alzheimer's disease. In embodiments, the method increases the blood or tissue level of thiamine in the subject compared to the blood or tissue level of thiamine in the subject before treatment. In embodiments, the method increases the blood or tissue level of thiamine to a pharmacological level effective to treat a neurodegenerative condition, such as dementia. In embodiments, the method increases the blood or tissue level of thiamine in the subject to a pharmacological level effective to reduce a symptom of a neurodegenerative condition, such as dementia. In an embodiment, the method increases the blood or tissue level of thiamine in the subject to a reference (normal or desired) level of tissue thiamine as described herein.

In some embodiments of the above method (third aspect), the subject may be administered at least 300 mg per day, or at least 600 mg per day, or at least 900 mg per day, or at least 1200 mg per day of any of the compounds described above, which may be a thiamine derivative such as encompassed by Formula (1). In some embodiments, the subject may be administered a daily dosage between 300-1500 mg, 600-1250 mg, 900-1200 mg, 900-1500 mg, or 1200-1500 mg of a compound of Formula (1) per day. In different embodiments, the daily dosage is achieved by two or three administrations. In different embodiments, the daily dosage may be administered for at least six, twelve, or eighteen months.

### BRIEF DESCRIPTION OF THE DRAWINGS

FIG. 1. Schematic summary of the treatment protocol for the one-year trial.

FIGS. 2A-2B. Changes in ADAS-cog with benfotiamine treatment compared to controls. FIG. 2A plots the ADAS-cog score per time while FIG. 2B plots the ADAS-cog score as change from baseline.

FIGS. 3A-3C. Benfotiamine treatment and the CDR. CDR Placebo=34, benfotiamine=29. FIG. 3A plots CDR score (changes from baseline) for placebo and benfotiamine, wherein the annotation “***” indicates significantly different (p=0.034) (A). FIG. 3B plots CDR score (changes from baseline) for placebo and benfotiamine for groups separated by sex (as shown, large but non-significant differences occur). FIG. 3C plots CDR score (changes from baseline) for placebo and benfotiamine for groups separated by presence of APOE4 allele (as shown, only the non-APOE ε4 allele group differs). In the non-APOE4 group the *** indicates values significantly different (p=0.013). The APOE4 denotes at least one e4 allele. P values here are when there are subgroups are all obtained from subgroup analysis, not interaction from ANOVA (C).

FIGS. 4A-4B. Benfotiamine and the Buschke Selective Reminding Test (SRT). FIG. 4A plots SRT score against event time (months) for placebo and benfotiamine, while FIG. 4B plots the SRT score as change from baseline.

FIGS. 5A-5C. Benfotiamine and the Neuropsychiatric Inventory (NPI). FIG. 5A (top panel) plots NPI score against even time (months) for placebo and benfotiamine, while FIG. 5A (bottom panel) plots the NPI score as change from baseline. As shown, no differences were seen in the overall scores. FIG. 5B plots month 12 baseline change in NPI score for placebo and benfotiamine, with separation of the groups by sex (the results revealed a highly significant benefit in males but not females), wherein *** indicates p=0.035. FIG. 5C plots month 12 baseline change in NPI score for placebo and benfotiamine, with separation of the groups by presence of APOE4 allele (as shown, no significant difference was seen with APO ε4 alleles).

FIGS. 6A-6B. Alzheimer's Disease Cooperative Study-Activities of Daily Living (ADCS-ADL). FIG. 6A plots ADCS-ADL score against event time (months) for placebo and benfotiamine, while FIG. 6B plots the ADCS-ADL score as change from baseline.

FIG. 7 plots blood thiamine, ThMP, and ThDP concentrations at baseline and month 12. Each dot represents a different patient. The bar represents the mean value. All values are per protocol after omitting a patient designated as placebo who was taking benfotiamine from another source.

FIG. 8 shows the relationship of sex and APOE ε4 genotype to thiamine, ThDP, and ThMP. Values are means±SEM. *** Denotes significantly different (p<0.0001) by t-test.

FIGS. 9A-9B. Advanced Glycation End Products (AGE) after benfotiamine treatment. The results were measured on serum and several samples were contaminated with RBC. In FIG. 9A, the n's are 12 placebo and 13 benfotiamine patients. The *** symbol indicates p=0.043. FIG. 9B plots the change of AGE for placebo and benfotiamine for APOE4 and non-APOE4 groups. In the APO e4 group, n=6. In the non-APOE4 group, n=7. The APO ε4 denotes at least one e4 allele.

FIG. 10A plots pattern score as function of the 12 month treatment period. The pattern is a linear combination of the first two Principal Components whose pattern score is slightly but significantly higher for treatment than untreated participants at time point of 12 months.

FIG. 10B (left and right panels). The left panel shows the pattern score plotted against CDR status (p-level obtained from whole-model F-test). A higher pattern score implies lower CDR status. The right panel shows loading distributions from a bootstrap test with 90% coverage intervals. Notably, these loading sizes and signs are relative since the whole-brain mean was removed from the analysis prior to the pattern derivation. Thus, high positive loadings are found in the right mid-temporal and inferior parietal cortex, implying relatively higher signal in participants with lower CDR. Bilateral cerebellum and paracentral lobule, on the other hand, had relatively lower signal in participants with lower CDR.

FIG. 10C (left and right panels). Stratification of the pattern score by APOE status reveals that APOE4 negative patients show the greatest response. APOE ε4=0 patients show a treatment effect (left panel), APOE ε4=1 do not (right panel).

### DETAILED DESCRIPTION

Disclosed herein are methods for treating a neurodegenerative disease in a subject, such as Alzheimer's Disease, by administering to the subject a pharmaceutically effective amount of a compound such as a synthetic derivative of thiamine, e.g., benfotiamine or a derivative thereof. The neurodegenerative disease may also be considered a neurodegenerative condition.

In one embodiment, the subject undergoing treatment has a neurodegenerative disease. In some embodiments, the neurodegenerative disease is characterized by cognitive impairment, as determined by a neuropsychological test or evaluation. The neuropsychological test or evaluation may be administered to the subject before or during the treatment. The cognitive impairment may, in turn, be characterized by any of the medically accepted criteria, e.g., memory loss or loss in problem-solving, reasoning, or comprehension, which is usually determined by administering a standardized assessment to the subject, e.g., the Alzheimer's Disease Assessment Scale-Cognitive Subscale (ADAS-Cog) test. The neurodegenerative disease may be, for example, Alzheimer's Disease (AD), Parkinson's Disease (PD), Huntington's Disease, multiple sclerosis (MS), amyotropic lateral sclerosis (ALS), or a prion disease. For any of the neurodegenerative diseases, the disease may be mild (early stage), middle (moderate stage), or severe (late stage). In particular embodiments, the neurodegenerative disease is mild, moderate, or severe AD, or mild, moderate, or severe cognitive impairment, or mild, moderate, or severe dementia.

In some embodiments, the neurodegenerative disease is characterized by one or more markers (such as a pathological marker) indicative of a neurodegenerative condition, the onset of a neurodegenerative condition, or a predisposition or likelihood of developing a neurodegenerative condition. The marker may be in the presence or absence of cognitive impairment in the subject, or more generally, in the presence or absence of symptoms (e.g., clinical or diagnosed symptoms) of the neurodegenerative disease. In some cases, a marker is pre-symptomatic (or asymptomatic). The marker may be, for example, amyloid plaques in the brain, neurofibrillary tangles in the brain, decline in brain glucose metabolism, decline in thiamine diphosphate-dependent enzyme activity in the blood or brain, and/or increase in advanced glycation end products in the blood, urine, or brain. The foregoing markers are particularly relevant to AD, but other markers relevant to other neurodegenerative diseases are considered herein. The subject may be tested by known testing methods for any of the foregoing markers, before or during the treatment (administration of the compound). In some embodiments, the subject is selected for treatment (administration of the compound) on the basis that the subject tests positive for the presence of one or more markers, such as one or more of those enumerated above, independent of or alternatively in addition to any clinical symptoms (such as any cognitive impairment).

In some embodiments, the neurodegenerative disease is characterized by amyloid plaques in the brain of the subject. In other embodiments, the neurodegenerative disease is characterized by neurofibrillary tangles in the brain of the subject. In other embodiments, the neurodegenerative disease is characterized by amyloid plaques and neurofibrillary tangles in the brain of the subject. In other embodiments, the neurodegenerative disease is characterized by a decline in glucose metabolism in the brain of the subject. In other embodiments, the neurodegenerative disease is characterized by a decline in thiamine diphosphate-dependent enzyme activity in the blood or brain of the subject. In other embodiments, the neurodegenerative disease is characterized by an increase in advanced glycation end products in the blood, urine, or brain of the subject. In particular embodiments, the neurodegenerative disease is Alzheimer's Disease, mild cognitive impairment (e.g., mild AD or mild dementia), or Parkinson's Disease.

In some embodiments, the subject is tested prior to treatment (administration of the compound) to determine the presence of one or more markers, such as one or more of those enumerated above, particularly for AD. In some embodiments, the subject is selected for treatment on the basis that the subject is positive for one or more markers, independent of or in addition to any clinical symptoms of a neurodegenerative disease (such as any cognitive impairment).

In one embodiment, the subject tests positive (or “is positive”) for at least one marker before and/or during the treatment (the administration of the compound). By testing positive for at least one marker, the subject is confirmed to possess or exhibit any one or more of the following conditions by appropriate testing: (i) amyloid plaques in the brain, (ii) neurofibrillary tangles in the brain, (iii) decline in brain glucose metabolism, (iv) decline in thiamine diphosphate-dependent enzyme activity in the blood or brain, and/or (v) increase in advanced glycation end products in the blood, urine, or brain. Notably, the test for a marker, whether direct or indirect, is based on testing of biological tissue (e.g., brain, blood, or urine) in a subject, and not based on a cognitive assessment of the subject. The test can be given before treatment, during treatment, or both.

In a first embodiment, the subject tests positive for amyloid plaques in the brain (the first listed marker) by any method capable of directly or indirectly detecting amyloid plaques in the brain, e.g., by amyloid PET scan with optional radiotracer (direct testing) or blood testing for amyloid protein (indirect testing). Detecting amyloid plaques by PET scan is well known. Blood testing for amyloid protein is described in, for example, Schindler S E, et al. High-precision plasma β-amyloid 42/40 predicts current and future brain amyloidosis. Neurology. 2019. Epub Aug. 1. doi: 10.1212/WNL.0000000000008081. If amyloid plaques are detected by PET scan, blood testing, or other method, the subject is considered to have tested positive for the first listed marker (amyloid plaques in the brain).

In a second embodiment, the subject tests positive for neurofibrillary tangles in the brain (the second listed marker) by any method capable of directly or indirectly detecting neurofibrillary tangles in the brain, e.g., by amyloid PET scan with optional radiotracer (direct testing). Detecting neurofibrillary tangles by PET scan is well known. For example, the measurement of phosphor-tau in plasma reflects tangles in the brain (S. Palmqvist et al. Prediction of future Alzheimer's disease dementia using plasma phospho-tau combined with other accessible measures. Nature Medicine. 2021; 27(6):1034-42). If neurofibrillary tangles are detected by PET scan or other method, the subject is considered to have tested positive for the second listed marker (neurofibrillary tangles in the brain).

In a third embodiment, the subject tests positive for decline in brain glucose metabolism (the third listed marker) by any method capable of directly or indirectly detecting a decline in brain glucose metabolism, e.g., by fluorine-18 (¹⁸F) fluorodeoxyglucose positron-emission tomography (FDG-PET), as well known in the art. A “decline” in brain glucose metabolism is determined to be present by comparing the test result for brain glucose metabolism with an earlier test result given to the subject for brain glucose metabolism. Nevertheless, in the event that an earlier test was not given, the term “decline” may also indicate that the brain glucose metabolism is less than an expected or known normal level. If a decline in brain glucose metabolism is detected by FDG-PET or other method, the subject is considered to have tested positive for the third listed marker (decline in brain glucose metabolism).

In a fourth embodiment, the subject tests positive for decline in thiamine diphosphate-dependent enzyme activity in the blood or brain (the fourth listed marker) by any method capable of directly or indirectly detecting a decline in thiamine diphosphate-dependent enzyme activity in the blood or brain, e.g., by testing of thiamine, thiamine diphosphate (ThDP), and/or thiamine monophosphate (ThMP) in the blood or brain, as well known in the art. A “decline” in thiamine diphosphate-dependent enzyme activity is determined to be present by comparing the test result for thiamine diphosphate-dependent enzyme activity with an earlier test result given to the subject for thiamine diphosphate-dependent enzyme activity. Nevertheless, in the event that an earlier test was not given, the term “decline” may also indicate that the thiamine diphosphate-dependent enzyme activity is less than an expected or known normal level. If a decline in thiamine diphosphate-dependent enzyme activity is detected by blood testing or other method, the subject is considered to have tested positive for the fourth listed marker (decline in thiamine diphosphate-dependent enzyme activity).

In a fifth embodiment, the subject tests positive for increase in advanced glycation end (AGE) products in the blood or brain (the fifth listed marker) by any method capable of directly or indirectly detecting an increase in AGE products in the blood, urine, or brain, e.g., by testing using a commercial kit for blood plasma sampling. AGE products may also be detected using a specific fluorescence assay on serum or urine, such as described in K. Yanagisawa et al., Metabolism, 47(11): 1348-1353, Nov. 1998, doi: 10.1016/s0026-0495(98)90303-1. An “increase” in AGE products is determined to be present by comparing the test result for level of AGE products with an earlier test result given to the subject for AGE products. Nevertheless, in the event that an earlier test was not given, the term “increase” may also indicate that the level of AGE products is more than an expected or known normal level. If an increase in AGE products is detected by blood or urine testing or other method, the subject is considered to have tested positive for the fifth listed marker (increase in AGE products).

The subject may test positive for the at least one marker before or after being tested for cognitive impairment, if cognitive impairment is tested. In another embodiment, the subject tests positive for at least one marker before or during the treatment and the subject also tests positive for cognitive impairment either before or after the subject tests positive for at least one marker. In another embodiment, the subject tests positive for at least one marker before or during the treatment and the subject tests negative for cognitive impairment (i.e., does not yet display cognitive impairment) either before or after the subject tests positive for at least one marker. In the latter case, the subject is considered asymptomatic (or pre-symptomatic) for the neurodegenerative disease but at risk for developing (e.g., likely to develop or having a pre-disposition to) the neurodegenerative disease. In one embodiment, the subject tests positive for at least one marker before or during the treatment (the administration of the compound) without being tested for cognitive impairment.

In some embodiments, after the subject tests positive for at least one marker, the subject is monitored during the treatment to determine whether the level of the marker changes (e.g., decrease or increase) during the treatment. The monitoring of the marker may or may not begin before the start of treatment. The administration of the compound (e.g., dosage, dosing schedule, selection of compound, and length of treatment) may be adjusted accordingly based on an observed change in the level of the marker. For example, in the case where the level of the marker is observed to improve (e.g., lessening of amyloid plaques or neurofibrillary tangles in the brain, or increase in glucose metabolism or thiamine diphosphate-dependent enzyme activity in the blood or brain, or decrease in AGE products in the blood, urine, or brain) during treatment, the administration of the compound may be accordingly decreased or even halted or terminated. Alternatively, in the case where the level of the marker is observed to remain the same or not improve during the administration of the compound, the administration of the compound may be accordingly maintained or increased (e.g., in dosage amount or length of treatment).

The subject being treated may or may not be a carrier of apolipoprotein E4 (apoE4). The subject can be determined to be a carrier of apoE4 by APOE genotype testing, as known in the art. The subject can be tested for the apoE4 gene before or during the treatment. In some embodiments, the subject is selected for treatment (administration of the compound) on the basis that the subject is not a carrier of apoE4. In some embodiments, the subject undergoes a cognitive test and/or a marker test in addition to (before or after) APOE genotype testing. In other embodiments, the subject undergoes APOE genotype testing and does not undergo a cognitive test or a marker test.

In the method, the subject is administered a pharmaceutically effective amount of a lipophilic or non-lipophilic derivative of thiamine, or more particularly benfotiamine or a derivative thereof, any of which are herein referred as the “compound” or “active compound”. In this regard, benfotiamine itself has limited lipophilicity. (See, e.g., Volvert M-L, Seyen S, Piette M, Evrard B, Gangolf M, Plumier J-C, et al. Benfotiamine, a synthetic S-acyl thiamine derivative, has different mechanisms of action and a different pharmacological profile than lipid-soluble thiamine disulfide derivatives. BMC Pharmacology. 2008; 8(1):10. PubMed PMID: doi:10.1186/1471-2210-8-10) The compound typically has a structure within the following generic structure:

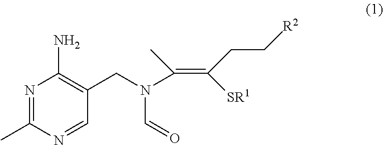

In Formula (1), R¹ is either —R or —SR, wherein R is a hydrocarbon group containing 1-20 carbon atoms. When R¹ is R, the compound of Formula (1) is a sulfide compound, such as benfotiamine. When R¹ is —SR, the compound of Formula (1) is a disulfide compound, such as found in allithiamine, fursultiamine, and sulbutiamine. In Formula (1), R² is independently selected from —OR′, —OPO₃²⁻, and —OC(O)R′, wherein R′ is a hydrogen atom or a hydrocarbon group containing 1-6 carbon atoms, which can be selected from any of the hydrocarbon groups (R) described above containing 1-6 carbon atoms. In some embodiments, R² is selected from —OR′ and —OC(O)R′. In some embodiments, R² is —OR′. In other embodiments, R² is —OC(O)R′. In other embodiments, R² is —OPO₃²⁻.

As used herein, the term “hydrocarbon group” (also denoted by the group R) is, in a first embodiment, composed solely of carbon and hydrogen. In different embodiments, one or more of the hydrocarbon groups can contain precisely, or a minimum of, or a maximum of, for example, one, two, three, four, five, six, seven, eight, nine, ten, eleven, twelve, thirteen, fourteen, fifteen, sixteen, seventeen, eighteen, nineteen, or twenty carbon atoms, or a number of carbon atoms within a particular range bounded by any two of the foregoing carbon numbers. Hydrocarbon groups in different compounds described herein, or in different positions of a compound, may possess the same or different number (or preferred range thereof) of carbon atoms in order to independently adjust or optimize the activity or other characteristics of the compound.

The hydrocarbon group (R) can be, for example, a saturated or straight-chained (i.e., linear) alkyl group. Some examples of straight-chained alkyl groups include methyl, ethyl, n-propyl, n-butyl, n-pentyl, n-hexyl, n-heptyl, n-octyl, n-nonyl, n-decyl, n-undecyl, n-dodecyl, n-tridecyl, n-tetradecyl, n-pentadecyl, n-hexadecyl, n-heptadecyl, n-octadecyl, and n-eicosyl groups.

The hydrocarbon group (R) can alternatively be a saturated and branched alkyl group. Some examples of branched alkyl groups include isopropyl (2-propyl), isobutyl (2-methylprop-1-yl), sec-butyl (2-butyl), t-butyl (1,1-dimethylethyl-1-yl), 2-pentyl, 3-pentyl, 2-methylbut-1-yl, isopentyl (3-methylbut-1-yl), 1,2-dimethylprop-1-yl, 1,1-dimethylprop-1-yl, neopentyl (2,2-dimethylprop-1-yl), 2-hexyl, 3-hexyl, 2-methylpent-1-yl, 3-methylpent-1-yl, isohexyl (4-methylpent-1-yl), 1,1-dimethylbut-1-yl, 1,2-dimethylbut-1-yl, 2,2-dimethylbut-1-yl, 2,3-dimethylbut-1-yl, 3,3-dimethylbut-1-yl, 1,1,2-trimethylprop-1-yl, and 1,2,2-trimethylprop-1-yl groups, isoheptyl, isooctyl, and the numerous other branched alkyl groups having up to 20 carbon atoms, wherein the “1-yl” suffix represents the point of attachment of the group.

The hydrocarbon group (R) can alternatively be a saturated and cyclic alkyl hydrocarbon group (i.e., cycloalkyl group). Some examples of cycloalkyl groups include cyclopropyl, cyclobutyl, cyclopentyl, cyclohexyl, cycloheptyl, and cyclooctyl groups. The cycloalkyl group can also be a polycyclic (e.g., bicyclic) group by either possessing a bond between two ring groups (e.g., dicyclohexyl) or a shared (i.e., fused) side (e.g., decalin and norbornane).

The hydrocarbon group (R) can alternatively be an unsaturated and straight-chained hydrocarbon group, i.e., straight-chained alkenyl (olefinic) or alkynyl group. The unsaturation occurs by the presence of one or more carbon-carbon double bonds and/or one or more carbon-carbon triple bonds. Some examples of straight-chained unsaturated groups include vinyl, propen-1-yl (allyl), 3-buten-1-yl (CH₂═CH—CH₂—CH₂—), 2-buten-1-yl (CH₂—CH═CH—CH₂—), butadienyl, 4-penten-1-yl, 3-penten-1-yl, 2-penten-1-yl, 2,4-pentadien-1-yl, 5-hexen-1-yl, 4-hexen-1-yl, 3-hexen-1-yl, 3,5-hexadien-1-yl, 1,3,5-hexatrien-1-yl, 6-hepten-1-yl, ethynyl, propargyl (2-propynyl), and the numerous other straight-chained alkenyl and alkynyl groups having up to 20 carbon atoms.

The hydrocarbon group (R) can alternatively be an unsaturated and branched hydrocarbon group, i.e., branched alkenyl or alkynyl groups. Some examples of branched unsaturated groups include propen-2-yl (CH₂=C·-CH₃), 1-buten-2-yl (CH₂=C·-CH₂—CH₃), 1-buten-3-yl (CH₂═CH—CH·-CH₃), 1-propen-2-methyl-3-yl (CH₂═C(CH₃)—CH₂—), 1-penten-4-yl, 1-penten-3-yl, 1-penten-2-yl, 2-penten-2-yl, 2-penten-3-yl, 2-penten-4-yl, and 1,4-pentadien-3-yl, wherein the dot in any of the foregoing groups indicates a point of attachment.

The hydrocarbon group (R) can alternatively be an unsaturated and cyclic hydrocarbon group, i.e., cycloalkenyl group. The unsaturated cyclic group can be aromatic or aliphatic. Some examples of unsaturated cyclic hydrocarbon groups include cyclopropenyl, cyclobutenyl, cyclopentenyl, cyclopentadienyl, cyclohexenyl, cyclohexadienyl, phenyl, benzyl, cycloheptenyl, cycloheptadienyl, cyclooctenyl, cyclooctadienyl, and cyclooctatetraenyl groups. The unsaturated cyclic hydrocarbon group can also be a polycyclic group (such as a bicyclic or tricyclic polyaromatic group) by either possessing a bond between two of the ring groups (e.g., biphenyl) or a shared (i.e., fused) side, as in naphthalene, anthracene, phenanthrene, phenalene, or indene fused ring systems.

One or more of the hydrocarbon groups (R) may (i.e., optionally) be substituted with (i.e., include) one or more heteroatoms, which are non-carbon non-hydrogen atoms. Some examples of heteroatoms include oxygen (O), nitrogen (N), sulfur (S), and halogen (halide) atoms. Some examples of halogen atoms include fluorine, chlorine, bromine, and iodine. In some embodiments, the heteroatom inserts between at least two carbon atoms (as in —C—O—C-ether, —C—S—C— thioether, —C—N(R)—C— tertiary amine, or —C═N—C— imine bonding systems) or between at least one carbon atom and at least one hydrogen atom (as in —C—OH, —C—SH, —C—NH₂, —C—NH—C—, or —C(═NH)C— bonding systems), wherein the shown carbon atom in each case can be considered part of a hydrocarbon group R described above. In other embodiments, the heteroatom replaces one or more hydrogen atoms and/or one or more carbon atoms in the hydrocarbon group, as in halogen-substituted groups (e.g., a —CH₂F, —CHF₂, —CF₃, or halogenated phenyl group) and carbonyl-substituted groups, such as ketone and aldehyde groups.

When two or more same or different heteroatoms are bound to each other or located on the same carbon atom, the resulting group containing the heteroatoms is herein referred to as a “heteroatom-containing group”. Some examples of heteroatom-containing groups include keto (—C(O)R″), carboxy (—C(O)OR″ or —OC(O)R″), thiocarboxy (—C(S)OR″ or —OC(S)R″), carboxamide (—C(O)NR″₂, —C(O)NR″—, or —N(R″)C(O)—), urea (—NR″—C(O)—NR″₂ or —NR″—C(O)—NR″—), thiourea (—NR″—C(S)—NR″₂ or —NR″—C(S)—NR″—), carbamate (—NR″—C(O)—OR″, —OC(O)—NR″₂, or —NR″—C(O)—O—), thiocarbamate (—NR″—C(S)—OR″, —OC(S)—NR″₂, or —NR″—C(S)—O—), nitro (NO₂), nitrile (CN), sulfonyl (—S(O)₂R″ or —S(O)₂—), sulfinyl (i.e., sulfoxide, —S(O)R″ or —S(O)—), disulfide (—C—S—S—C—), sulfonate (—S(O)₂R″), and amine oxide (as typically found in a nitrogen-containing ring), wherein R″ independently represents hydrogen atom or any of the hydrocarbon groups (R) described above. For example, —C(O)OR″ includes carboxylic acid (—C(O)OH) and carboxylic ester (—C(O)OR), where R is any of the hydrocarbon groups described above. The heteroatom-containing group may also either insert between carbon atoms or between a carbon atom and hydrogen atom, if applicable, or replace one or more hydrogen and/or carbon atoms.

In some embodiments, the hydrocarbon group (R) is substituted with one or more halogen atoms to result in a partially halogenated or perhalogenated hydrocarbon group. Some examples of partially halogenated hydrocarbon groups include —CHY₂, —CH₂Y, —CH₂CY₃, —CH(CY₃)₂, or a halo-, dihalo-, trihalo-, or tetrahalo-substituted phenyl group, wherein Y represents any of F, Cl, Br, or I, and more commonly F or Cl. Some examples of perhalogenated hydrocarbon groups include —CY₃, —CY₂CY₃, —CY₂CY₂CY₃, —CY(CY₃)₂, or perhalophenyl (—C₆Y₅).

In some embodiments, the hydrocarbon group (R) is, or includes, a cyclic or polycyclic (i.e., bicyclic, tricyclic, or higher cyclic) saturated or unsaturated (e.g., aliphatic or aromatic) hydrocarbon group that includes at least one ring heteroatom, such as one, two, three, four, or higher number of ring heteroatoms. Such heteroatom-substituted cyclic hydrocarbon groups are referred to herein as “heterocyclic groups”. As used herein, a “ring heteroatom” is an atom other than carbon and hydrogen (typically, selected from nitrogen, oxygen, and sulfur) that is inserted into or replaces a ring carbon atom in a hydrocarbon ring structure. In some embodiments, the heterocyclic group is saturated. In other embodiments, the heterocyclic group is unsaturated, i.e., aliphatic or aromatic heterocyclic groups, wherein the aromatic heterocyclic group is also referred to herein as a “heteroaromatic ring”, or a “heteroaromatic fused-ring system” in the case of at least two fused rings, at least one of which contains at least one ring heteroatom.

Some examples of saturated heterocyclic groups containing at least one oxygen atom include oxetane, tetrahydrofuran, tetrahydropyran, 1,4-dioxane, 1,3-dioxane, and 1,3-dioxepane rings. Some examples of saturated heterocyclic groups containing at least one nitrogen atom include pyrrolidine, piperidine, piperazine, imidazolidine, azepane, and decahydroquinoline rings. Some examples of saturated heterocyclic groups containing at least one sulfur atom include tetrahydrothiophene, tetrahydrothiopyran, 1,4-dithiane, 1,3-dithiane, and 1,3-dithiolane rings. Some examples of saturated heterocyclic groups containing at least one oxygen atom and at least one nitrogen atom include morpholine and oxazolidine rings. An example of a saturated heterocyclic group containing at least one oxygen atom and at least one sulfur atom includes 1,4-thioxane. An example of a saturated heterocyclic group containing at least one nitrogen atom and at least one sulfur atom includes thiazolidine and thiamorpholine rings.

Some examples of unsaturated heterocyclic groups containing at least one oxygen atom include furan, pyran, 1,4-dioxin, benzofuran, dibenzofuran, and dibenzodioxin rings. Some examples of unsaturated heterocyclic groups containing at least one nitrogen atom include pyrrole, imidazole, pyrazole, pyridine, pyrazine, pyrimidine, 1,3,5-triazine, azepine, diazepine, indole, purine, benzimidazole, indazole, 2,2′-bipyridine, quinoline, isoquinoline, phenanthroline, 1,4,5,6-tetrahydropyrimidine, 1,2,3,6-tetrahydropyridine, 1,2,3,4-tetrahydroquinoline, quinoxaline, quinazoline, pyridazine, cinnoline, 5,6,7,8-tetrahydroquinoxaline, 1,8-naphthyridine, and 4-azabenzimidazole rings. Some examples of unsaturated heterocyclic groups containing at least one sulfur atom include thiophene, thianaphthene, benzothiophene, thiochroman, and thiochromene rings. Some examples of unsaturated heterocyclic groups containing at least one oxygen atom and at least one nitrogen atom include oxazole, isoxazole, benzoxazole, benzisoxazole, oxazoline, 1,2,5-oxadiazole (furazan), and 1,3,4-oxadiazole rings. Some examples of unsaturated heterocyclic groups containing at least one nitrogen atom and at least one sulfur atom include thiazole, isothiazole, benzothiazole, benzoisothiazole, thiazoline, and 1,3,4-thiadiazole rings.

In some embodiments of Formula (1), R¹ is ether —R or —SR wherein R is an unsaturated hydrocarbon group, as described above, or more particularly, a phenyl or benzyl (—CH₂-phenyl) group, wherein the phenyl in either group may or may not be substituted with one, two, or three halogen atoms or heteroatom-containing groups as described above. In other embodiments, R (or more specifically, R¹) is —C(O)R^{a}, wherein R^{a} is any of the hydrocarbon groups containing 1-20 carbon atoms and optionally containing one or more heteroatoms selected from oxygen, nitrogen, and sulfur, as described above. In particular embodiments, R^{a} is an unsaturated hydrocarbon, or more particularly, a phenyl or benzyl group, which may or may not be substituted with any one or more heteroatoms or heteroatom-containing groups described above. In separate or further embodiments of Formula (1), R² is —OPO₃²⁻ (typically, —OPO₃H₂), —OH, OR, or —OC(O)R′, or more particularly —OPO₃²⁻ or —OPO₃H₂. Notably, when R¹ is —C(O)R^{a} with R^{a} being phenyl, and when R² is —OPO₃²⁻, the resulting compound corresponds to benfotiamine, or a derivative thereof if the phenyl group is derivatized with one or more with any one or more heteroatoms or heteroatom-containing groups described above.

The compound being administered may, in some embodiments, be in the form of a pharmaceutically acceptable salt. The pharmaceutically acceptable salt can result from reaction of the neutral compound with an electrophilic organic species or a pharmaceutically acceptable organic or inorganic acid. Some examples of electrophilic organic species include the alkyl halides, such as methyl bromide, ethyl bromide, n-propyl bromide, and isopropyl bromide. Some examples of organic acids include acetic acid, propionic acid, butanoic acid, glycolic acid, pyruvic acid, lactic acid, malonic acid, malic acid, and citric acid. Some examples of inorganic acids include hydrochloric acid, hydrobromic acid, hydroiodic acid, sulfuric acid, nitric acid, phosphoric acid, and metaphosphoric acid. In the case of benfotiamine and its derivatives, the primary amine group may be reacted with any of the foregoing acids or electrophilic organic species to produce a salt of the compound. The salt may also result from reaction of the neutral compound with a pharmaceutically acceptable base. In the case of benfotiamine and its derivatives, the phosphonic acid group may react with a base (e.g., NaOH) to form a salt (e.g., sodium salt).

The compound being administered may, in some embodiments, be in the form of a solvate. As known in the art, a solvate is an adduct of a compound with one or more solvent molecules. For purposes of the present invention, the solvent molecule should be pharmaceutically acceptable. Some examples of pharmaceutically acceptable solvent molecules include water, alcohols (e.g., ethanol), and glycols (e.g., ethylene glycol and propylene glycol). In the case of the solvent molecule being water, the solvate is typically referred to as a hydrate.

The compound being administered may, in some embodiments, be in the form of a polymorph. The polymorphic form may be, e.g., amorphous, single crystalline, or polycrystalline. The crystalline form may also be one of several possible crystalline forms governed by, for example, the crystal packing and crystallographic (symmetry) space group. Pharmaceutical solvates, hydrates, polymorphs, and crystalline forms are described in, for example, A. M. Healy et al., Advanced Drug Delivery Reviews, 117, 25-46, 2017 and S. L. Morissette et al., Advanced Drug Delivery Reviews, 56, 275-300, 2004, the contents of which are herein incorporated by reference in their entirety.

Notably, in all instances throughout this disclosure where the term “compound” or “a compound of Formula (1)” appears, any of these terms are understood to be inclusive of salts, solvates, and polymorphs as described above, unless specifically stated otherwise. In some embodiments, if desired, the compound may not include salt forms, solvate forms, or polymorphic forms.

In particular embodiments, the compound of Formula (1) is or includes benfotiamine, or a pharmaceutically acceptable salt, solvate, or polymorph thereof. The general structure of benfotiamine is provided as follows:

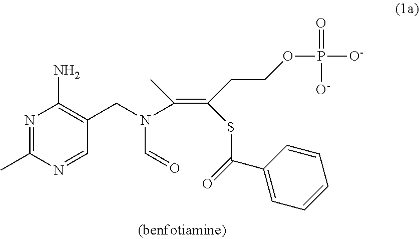 [SMILES: C/C(=C(\CCOP(=O)([O-])[O-])SC(=O)c1ccccc1)N(C=O)Cc1cnc(C)nc1N]

Although the structure of benfotiamine is depicted above as containing a double negative charge on the phosphate group, benfotiamine is typically doubly protonated, i.e., as the phosphoric acid group (—OPO₃H₂). Nevertheless, the anionic structure, as depicted, is meant to include salts of benfotiamine in which one or both of the protons on the phosphate group is replaced with another cationic species, such as sodium, potassium, or ammonium. Alternatively, benfotiamine may be in a protonated ammonium salt form.

Some examples of compounds according to Formula (1) other than benfotiamine include the following:

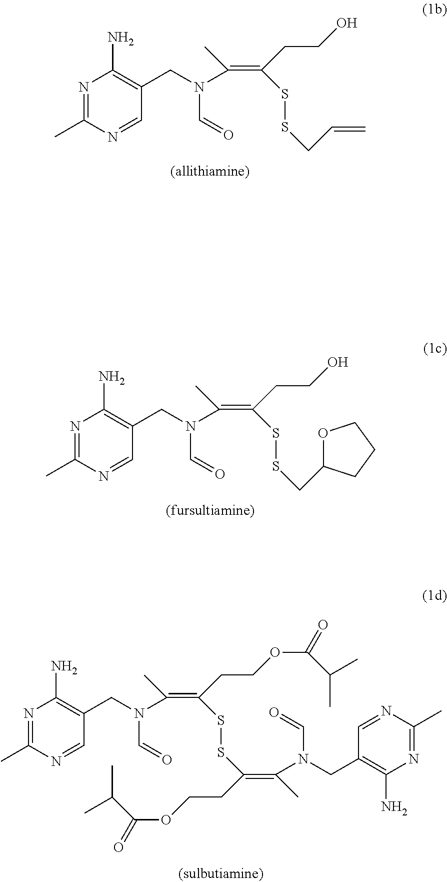

In an embodiment, the compound of Formula (1) is or includes a Z-isomer of benfotiamine. In some embodiments, the compound of Formula (1) excludes an E-isomer of benfotiamine. In an embodiment, the compound of Formula (1) is sulbutiamine. In an embodiment, the compound of Formula (1) does not comprise a monophosphate group.

The compounds described herein according to Formula (1) are either commercially available or can be synthesized according to methodologies and techniques well known in the art. For example, derivatives of benfotiamine in which the phenyl ring is substituted with halogen atoms or heteroatom-containing groups (e.g., nitro, cyano, sulfo, amino, carboxyl, hydroxyl, thiol, alkoxy, acyl, and hydrocarbon groups) may be synthesized by methods described in U.S. Application Pub. 2021/0163515, the contents of which are incorporated herein by reference.

In another aspect, the invention is directed to pharmaceutical compositions that contain any of the above-described compounds according to Formula (1) dispersed in a pharmaceutically acceptable carrier, i.e., vehicle or excipient. The compound being administered is typically in the form of a pharmaceutical composition containing the compound admixed with, dispersed within, or dissolved in a pharmaceutically acceptable carrier. The compound is dispersed in the pharmaceutically acceptable carrier by either being mixed (e.g., in solid form with a solid carrier) or dissolved or emulsified in a liquid carrier. The pharmaceutical composition may or may not also be formulated together with one or more additional active ingredients or adjuvants that improve the overall efficacy of the pharmaceutical composition, particularly as relates to the treatment of neurodegenerative disease.

The compound may be formulated into pharmaceutical compositions and dosage forms according to methods well known in the art. The pharmaceutical compositions of the present invention may be specially formulated for administration in liquid or solid form. The pharmaceutical formulation may be formulated to be suitable for any type of administration, such as oral administration (e.g., as tablets, capsules, powders, granules, pastes, solutions, suspensions, drenches, or syrups); parenteral administration (e.g., by subcutaneous, intramuscular or intravenous injection as provided by, for example, a sterile solution or suspension); topical application (e.g., as a cream, ointment, or spray); sublingual or buccal administration; transdermal administration; or nasal administration.

The phrase “pharmaceutically acceptable” refers herein to those substances, materials, compositions, and/or dosage forms which are, within the scope of sound medical judgment, suitable for administration to a subject. The phrase “pharmaceutically acceptable carrier,” as used herein, refers to a pharmaceutically acceptable vehicle, such as a liquid or solid filler, diluent, carrier, manufacturing aid (e.g., lubricant, talc magnesium, calcium or zinc stearate, or stearic acid), solvent, or encapsulating material, that serves to carry the therapeutic composition for administration to the subject. Each carrier should be “acceptable” in the sense of being compatible with the other ingredients of the formulation and physiologically safe to the subject. Any of the carriers known in the art can be suitable herein depending on the mode of administration.

Some examples of materials that can serve as pharmaceutically acceptable excipients, particularly for liquid forms, include water; isotonic saline; pH buffering agents; sugars (e.g., lactose, glucose, sucrose, and oligosaccharides, such as sucrose, trehalose, lactose, or dextran); and antimicrobials. Other excipients, more typically used in solid dosage forms, may also be included, e.g., starches (e.g., corn and potato starch); cellulose and its derivatives (e.g., sodium carboxymethyl cellulose, ethyl cellulose and cellulose acetate); gelatin; talc; waxes; oils (e.g., peanut oil, cottonseed oil, safflower oil, sesame oil, olive oil, corn oil and soybean oil); glycols (e.g., ethylene glycol, propylene glycol, and polyethylene glycol); polyols (e.g., glycerin, sorbitol, and mannitol); esters (e.g., ethyl oleate and ethyl laurate); agar; and other non-toxic compatible substances employed in pharmaceutical formulations. If desired, certain sweetening and/or flavoring and/or coloring agents may be added. Other suitable excipients can be found in standard pharmaceutical texts, e.g., in “Remington's Pharmaceutical Sciences”, The Science and Practice of Pharmacy, 19th Ed. Mack Publishing Company, Easton, Pa., (1995).

In some embodiments, the carrier further includes a molecular or microscopic (e.g., microscale or nanoscale) sub-carrier in which the compound is loaded, either within and/or conjugated onto the surface of the sub-carrier. The sub-carrier can be composed of, for example, a biocompatible and biodegradable polymer, e.g., based on a polyhydroxyacid biopolyester or polysaccharide. The overall structure of the sub-carrier can be, for example, a micelle, a liposome, dendrimer, nanoparticle, or porous scaffold. These and numerous other types of sub-carriers are well known in the art. The sub-carrier may function to protect the compound during transit, e.g., while in the bloodstream or while passing through the gastrointestinal tract, to release the compound closer to the target cells with lower chance of degradation. The carrier may also function to regulate the rate of release of the compound, such as delayed release or time release. The sub-carrier may also be functionalized with one or more targeting agents that selectively target a class of cells or biological molecules or proteins to be treated with the compound, such as specific receptors in the brain.

In embodiments, the compound according to Formula (1) is administered via an extended release formulation (e.g., sustained release or controlled release) with a release rate, within a predetermined range, of the compound into a biological fluid of the subject. In embodiments, the extended release within a predetermined range of the compound substantially maintains a minimum predetermined concentration in the biological fluid of the subject within a predefined amount of time. In embodiments, the extended release formulation administered to a subject effects a substantially constant release rate of the compound within the subject over a predefined amount of time. In embodiments, 300 mg, 600 mg, 900 mg, or 1200 mg of the compound are released within the subject over a period of 24 hours. In embodiments, greater than 1200 mg of compound are released within the subject over a period of 24 hours. In embodiments, the extended release formulation is a tablet, capsule, microcapsule, micelle, or liposome. In embodiments, extended release is effected via chemical means. In embodiments, extended release is effected via physical means. In embodiments, extended release is effected via both chemical and physical means.

In the method, any of the above described compounds, typically as a pharmaceutical formulation, is administered to the subject in a pharmaceutically (i.e., therapeutically) effective amount to treat a disease or condition. As is well known in the art, the dosage of the active ingredient(s) depends on such factors as the type and stage of the neurodegenerative condition, the method of administration, size of the patient, and potential side effects. Dosing is dependent on the severity and responsiveness of the neurodegenerative condition being treated, with the course of treatment lasting from several days, weeks, to several months, or until an improvement is observed, or symptoms have diminished, or a statis or diminution of the condition is achieved. Optimal dosing schedules can be calculated from measurements of drug accumulation in the body of the patient. The administering physician can determine optimal dosages, dosing methodologies, and repetition rates.

In different embodiments, depending on the above and other factors, the dosage of the compound according to Formula (1) being administered to a patient may be precisely, at least, or above, for example, 300 mg, 400 mg, 500 mg, 600 mg, 700 mg, 800 mg, 900 mg, 1000 mg, 1100 mg, 1150 mg, 1200 mg, 1250 mg, 1300 mg, 1350 mg, 1400 mg, 1450 mg, or 1500 mg per day, wherein the patient typically weighs 40-100 kg, or more typically, 50 kg, 60 kg, or 70 kg. The dosage per day may also be within a range bounded by any of the foregoing exemplary dosages, e.g., 300-1500 mg/day, 600-1500 mg/day, 900-1500 mg/day, 1000-1500 mg/day, 1200-1500 mg/day, 300-1250 mg/day, 600-1250 mg/day, 900-1250, 1000-1250 mg/day, 300-1200 mg/day, 600-1200 mg/day, 900-1200, or 1000-1200 mg/day. The composition may be administered once, twice, three times, or four times per day to achieve the indicated dosage. For example, a 300 mg/day dosage may be achieved by administering two doses of 150 mg per day or three doses of 100 mg per day. As another example, a 600 mg/day dosage may be achieved by administering two doses of 300 mg per day, or three doses of 200 mg per day, or four doses of 150 mg per day. As another example, a 900 mg/day dosage may be achieved by administering two doses of 450 mg per day, or three doses of 300 mg per day, or four doses of 225 mg per day. As another example, a 1200 mg/day dosage may be achieved by administering two doses of 600 mg per day, or three doses of 400 mg per day, or four doses of 300 mg per day. In some embodiments, benfotiamine or a derivative thereof is administered according to any of the above dosages or schedules.

Any of the daily dosages described above may be administered by any suitable schedule (i.e., frequency or regimen) over the course of the treatment, such as every day (daily), every other day, every third day, once per week, twice per week, or three times per week. The total treatment time over which the compound of Formula (1) is administered may be, for example, precisely or at least one month, two months, three months, four months, five months, six months, nine months, twelve months, fifteen months, eighteen months, or twenty-four months. Alternatively, or in addition, the composition is administered until a desired change is evidenced, either by an observed improvement in cognitive ability or level of pathological marker in the blood or brain.

In a first particular embodiment, the subject is administered at least or greater than 300 mg of a compound of Formula (1) per day for at least six months.

In a second particular embodiment, the subject is administered at least or greater than 300 mg of a compound of Formula (1) per day for at least twelve months.

In a third particular embodiment, the subject is administered at least or greater than 300 mg of a compound of Formula (1) per day for at least eighteen months.

In a fourth particular embodiment, the subject is administered at least or greater than 600 mg (e.g., as one dose or two doses of 300 mg or more per day) of a compound of Formula (1) per day for at least six months.

In a fifth particular embodiment, the subject is administered at least or greater than 600 mg (e.g., as one dose or two doses of 300 mg ore more per day) of a compound of Formula (1) per day for at least twelve months.

In a sixth particular embodiment, the subject is administered at least or greater than 600 mg (e.g., as one dose or two doses of 300 mg or more per day) of a compound of Formula (1) per day for at least eighteen months.

In a seventh particular embodiment, the subject is administered at least or greater than 900 mg (e.g., as one dose, two doses, or three doses (of 300 mg or more) per day) of a compound of Formula (1) per day for at least six months.

In an eighth particular embodiment, the subject is administered at least or greater than 900 mg (e.g., as one dose, two doses, or three doses (of 300 mg or more) per day) of a compound of Formula (1) per day for at least twelve months.

In a ninth particular embodiment, the subject is administered at least or greater than 900 mg (e.g., as one dose, two doses, or three doses of (300 mg or more) per day) of a compound of Formula (1) per day for at least eighteen months.

In a tenth particular embodiment, the subject is administered at least or greater than 1200 mg (e.g., as one dose, two doses, three doses, or four doses (of 300 mg or more) per day) of a compound of Formula (1) per day for at least six months.

In an eleventh particular embodiment, the subject is administered at least or greater than 1200 mg (e.g., as one dose, two doses, three doses, or four doses (of 300 mg or more) per day) of a compound of Formula (1) per day for at least twelve months.

In a twelfth particular embodiment, the subject is administered at least or greater than 1200 mg (e.g., as one dose, two doses, three doses, or four doses (of 300 mg or more) per day) of a compound of Formula (1) per day for at least eighteen months.

In embodiments, the dosages are administered by an extended release formulation. In embodiments, the dosages are administered by an implanted extended release formulation, which effects a daily dosage as described herein for every day over a period of 3 months, 6 months, 12 months (one year), 18 months, 24 months, 36 months, or 48 months. In some embodiments, the compound is administered daily for at least five years or for at least ten years. In some embodiments, the compound is administered daily for the duration of the subject's life after initial treatment. In embodiments, the dosages are administered by an implanted extended release formulation which effects a daily dosage as described herein for every day over a period in excess of one year. In embodiments, the extended release formulation is implanted in the subject. In embodiments, the extended release formulation is injected into the subject. In further embodiments, the extended release formulation is injected into the subject subcutaneously or intramuscularly.

In some embodiments, cognitive improvement is observed during (e.g., at 3, 6, 9, 12, 15, 18, 24, or 36 months) or at the completion of the treatment, such as any of the treatment regimens provided above. The level of amyloid plaques and/or neurofibrillary tangles may also be reduced as a result of the treatment. Normal glucose metabolism or thiamine diphosphate-dependent enzyme activity in the brain may alternatively or in addition be improved or restored as a result of the treatment. The level of advanced glycation end (AGE) products in the brain or blood may alternatively or in addition be lowered as a result of the treatment.

Any of the compounds according to Formula (1) described above may (i.e., optionally) be co-administered with one or more other therapeutic agents outside the scope of Formula (1). In a first instance, the co-administration is accomplished by including a compound of Formula (1) in admixture with one or more other therapeutic agents in the same pharmaceutical composition being administered. In a second instance, the co-administration can be accomplished by administering a compound of Formula (1) separately from one or more other therapeutic agents, i.e., at the same time or at different times. The other therapeutic agent may be, for example, an amyloid beta-directed monoclonal antibody (e.g., aducanumab), cholinesterase inhibitor (e.g., donepezil, rivastigmine, or galantamine), glutamate regulator (e.g., memantine), or orexin receptor antagonist (e.g., suvorexant). In some embodiments, the compound of Formula (1) is not administered with one or more of another therapeutic agent, such as those described above, or the compound of Formula (1) is the sole active compound being administered for treating the neurodegenerative disease.

In another aspect, the present disclosure is directed to a method of increasing a peripheral blood level or tissue level of thiamine in a subject having a decreased peripheral blood level or tissue level of thiamine, wherein the decreased peripheral blood level or tissue level of thiamine is not due to a dietary insufficiency of thiamine. In some cases, the subject (particularly those with AD) may show a tissue deficiency of thiamine without showing reduced thiamine in the blood. The method includes administering to the subject a pharmaceutically effective amount of a compound of Formula (1) as described above. Any of the daily dosages, schedules, and lengths of treatment further described and exemplified throughout this disclosure may be used to increase a peripheral blood level or tissue level of thiamine in a subject. The subject being treated for thiamine blood or tissue deficiency may or may not have a neurodegenerative disease and may or may not display symptoms of cognitive decline and may nor may not be positive for the presence of one or more markers described above. The subject may also be monitored before, during, and/or after the treatment to determine whether the peripheral blood or tissue level of thiamine has improved. If an improvement in peripheral blood or tissue level of thiamine is observed during the treatment, the administration of the compound may be appropriately adjusted as described earlier above.

In some embodiments, administering to the subject a pharmaceutically effective amount of the compound according to Formula (1) also increases a peripheral blood or tissue level of thiamine diphosphate and of thiamine monophosphate in the subject. In some embodiments, the compound is administered daily for a period of at least or more than six, eight, ten, twelve, eighteen, or twenty-four months, and wherein the peripheral blood or tissue level of thiamine in the subject at the end of treatment is increased at least or more than 10-fold, 20-fold, 50-fold, 80-fold, or 100-fold relative to the peripheral blood or tissue level of thiamine prior to treatment. In some embodiments, the compound is administered daily for a period of at least or more than six, eight, ten, or twelve months, and wherein the peripheral blood or tissue level of thiamine in the subject at the end of treatment is increased at least or more than one of 120-fold, 140-fold or 160-fold relative to the peripheral blood or tissue level of thiamine prior to treatment.

In embodiments of the methods described herein, the subject is human. In embodiments, the subject does not have diabetes. In embodiments, the subject does not have diabetes. In embodiments, the subject does not have prediabetes. In embodiments, the subject has not been diagnosed as diabetic. In embodiments, the subject does not have a peripheral neuropathy. In embodiments, the subject does not have a diabetic peripheral neuropathy. In embodiments, the subject has not been diagnosed with a diabetic peripheral neuropathy. In embodiments, the subject has not been diagnosed with a peripheral neuropathy associated with chemotherapy. In embodiments, the subject has not undergone, or is not presently undergoing a chemotherapy. In embodiments, the subject does not have a cancer, and/or has not been diagnosed with a cancer.

In embodiments of the methods described herein, the subject has a dementia, and the method increases peripheral thiamine in the subject. In embodiments of the methods described herein, the subject has diabetes, and a dementia, and the method increases peripheral thiamine in the subject.

In embodiments, the subject has not been diagnosed with Alzheimer's disease. In embodiments, the subject has mild cognitive impairment. In embodiments, the subject has Alzheimer's disease. In embodiments, the subject has Alzheimer's disease and is deemed or assessed incapable of taking one or more daily doses of the compound over a week, a month, 3 months, 6 months or a year. In embodiments, the subject has Alzheimer's disease and is deemed or assessed incapable of taking one or more daily doses of the compound over a week, a month, 3 months, 6 months or a year and is administered the compound in an extended release formulation which is implanted or injected into the subject. In an embodiment, the methods herein further comprise assessing if the subject is capable, or likely capable, of taking one or more daily doses of the compound over a week, a month, 3 months, 6 months, 12 months (a year), 18 months, or 24 months, and administering to a subject assessed as incapable or likely incapable the compound in an extended release formulation which is implanted or injected into the subject. In embodiments, the implanted or injected extended release formulation effects a daily dosage as described herein for every day over a period of 3 months, 6 months, 12 months (a year), 18 months, or 24 months. In embodiments, the implanted or injected extended release formulation effects a daily dosage as described herein for every day over a period in excess of one year.

Examples have been set forth below for the purpose of illustration and to describe the best mode of the invention at the present time. However, the scope of this invention is not to be in any way limited by the examples set forth herein.

### Examples

Overview, Materials, and Methods

The aim of this study was to conduct a double-blind early phase II randomized placebo-controlled trial of benfotiamine with the objective of collecting preliminary data on feasibility, safety, and efficacy. The goal was to test whether benfotiamine treatment could delay clinical decline in amyloid positive patients with amnestic mild cognitive impairment (aMCI) or mild dementia due to AD with MMSE scores of >21. ADAS-cog served as the primary endpoint. Brain glucose utilization, measured using FDG PET imaging, was assessed as a secondary endpoint. Cerebral glucose metabolism declines in temporoparietal regions with the progression of AD, correlates with clinical decline, and is also a sensitive measure of changes in regional neuronal function associated with disease or treatment effect (B. A. Gordon et al., The Lancet Neurology, 17, 241-250, 2018). AGE levels were used as a peripheral marker of efficacy. Measures of thiamine and its esters thiamine diphosphate (ThDP) and thiamine monophosphate (ThMP) provided blood markers of efficacy of drug delivery.

Patient population. Seventy amyloid positive patients 60 years and older with aMCI (21<MMSE<26) or mild AD dementia (MMSE≥26) were included. Table 1 (below) shows the inclusion and exclusion criteria.

TABLE 1
Inclusion and Exclusion Criteria

Inclusion criteria. Each patient met the following criteria:
Subjects who are able and willing to provide informed consent.
Male and non-pregnant, non-lactating, postmenopausal, or surgically sterilized female
subjects at least 60 years of age or older.
Clinical diagnosis of amnestic MCI by the Peterson criteria or probable AD dementia
according to the National Institute of Neurological Disorders and stroke and the
Alzheimer's Disease related Disorders Association (NINCDS/ADRDA)
MMSE score >21, CDR score >0.5 and <1 Cornell Scale for Depression in Dementia
(CSDD) score <10.
Ambulatory or ambulatory with aide
Has a caregiver willing to accompany the patient to each visit, accept responsibility for
supervising treatment and provided input to clinical outcome assessments
Reside at home
Speak English
Amyloid positive PET-scan
Patients taking FDA approved medications for the treatment of Alzheimer's Disease
[e.g., donepezil (Aricept), galantamine (Razadyne), rivastigmine (Exelon), or memantine
(Namenda)] for three months prior to baseline. Patients not on these medications did not
initiate them during the study.
Exclusion criteria
Significant neurological disorder other than AD including hypoxia, stroke, traumatic brain
injury
A current psychiatric disorder according to the DSM-IV diagnosis of major depression
unless successfully treated on a stable dose of an antidepressant for at least 4 weeks and
continues on stable dose throughout the study
Any other DSM-IV Axis 1 diagnosis including other primary neurodegenerative dementia,
schizophrenia or bipolar depression
A current diagnosis of uncontrolled Type I or Type II diabetes mellitus [Hemoglobin A1C
(Hb A1C <8]. Patients with uncontrolled diabetes (i.e., if glucose values exceed 200
mg/ml.
A current diagnosis of active, uncontrolled seizure disorder.
A current diagnosis of probable or possible vascular dementia according to NINDS-
AIREN .
An investigational drug during the previous 4 weeks.
Any previous exposure to Benfotiamine.
A current diagnosis of severe unstable cardiovascular disease.
A current diagnosis of acute severe, or unstable asthmatic condition (e.g., severe chronic
obstructive pulmonary disease (COPD),
A current diagnosis of cardiac, renal or hepatic disease.
A current diagnosis of cancer including any active treatment.
History of alcoholism, current or within past 5 years.
A disability that may prevent the patient from completing all study requirements (e.g.,
blindness, deafness, severe language difficulty).

Sample size justification. In addition to literature that states a four-point change on the ADAS-Cog is considered clinically significant, several randomized clinical trials have found ADAS-Cog change scores differed by 3-4 points between placebo and treatment groups over a 6-month time period. Moreover, other studies report annual changes in the ADAS-Cog among those who are untreated to average 9.6 points (SD=8.2) (K. Rockwood et al., BMC Neurology, 7, 26, 2007). Power was calculated based on the expected difference in change on the ADAS-COG of 3 points between the treatment and control groups. Estimates based on using a two-sided alpha of 0.05 and a standard deviation of 4, enrolling 29 patients per group, (N=58) suggest 80% power to detect a mean change of 3 between treatment and placebo.

Assignment of patients. A randomized, placebo-controlled, double-blinded trial of benfotiamine in persons with aMCI or AD dementia with a duration of 12 months was conducted. Using blocked, stratified randomization design, patients were assigned to the treatment or control group. By the inclusion criteria all subjects had MMSE of >21. Within this group, a separate randomization schedule was generated using the proc plan function in SAS statistics program for those with an MMSE greater than or less than or equal to 26 to balance their allocation patients to placebo or treatment groups. Using a block size of four for a total of seventy-six patients, 19 blocks were created to help ensure balanced recruitment into treatment and control groups within strata. The schedule was generated in advance by the statistician and provided to the blinded pharmacist in charge of executing the randomization. Two randomization worksheets stratified by MMSE were provided to the pharmacist, who randomized the patients. One sheet had MMSE Scores >=26 (randomized to Active or Placebo). The other sheet had MMSE scores <26. The patients were enrolled by the clinical study team and randomized by the pharmacist. The assignment to the treatment or placebo group was known only to the pharmacist and kept behind a triple lock. The patients received numbered bottles.

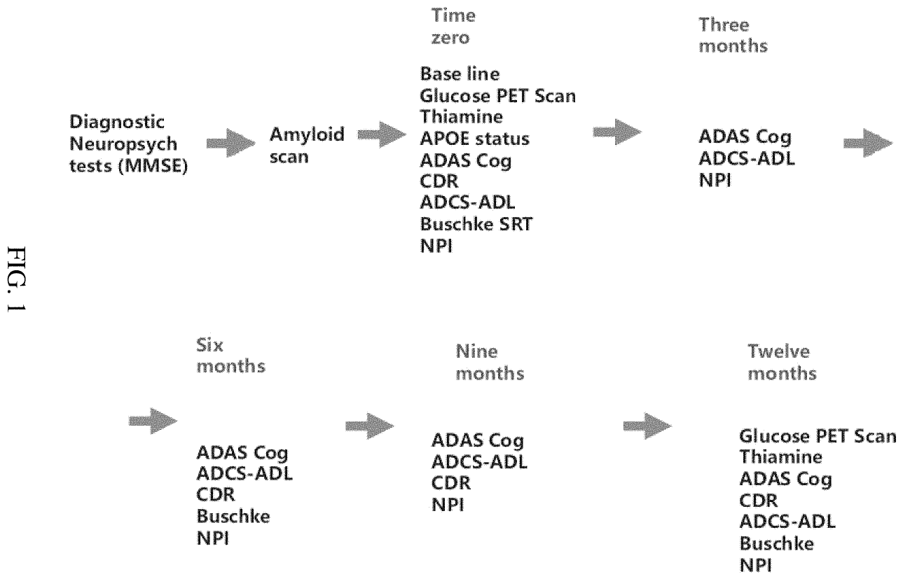

Study procedures. The trial was conducted as shown in FIG. 1 and registered in ClinicalTrials.gov (NCT02292238). Participants were pre-screened from the database of the Memory Evaluation Treatment Service (METS) at Burke Rehabilitation Center or referrals from the Center for the Aging Brain (CAB) at Montefiore/Einstein Medical College, Alzheimer's Association, primary care physicians, and private neurologists from the lower Hudson Valley region. aMCI or mild AD dementia were diagnosed according to NIA-AA workgroups criteria (G. M. McKhann et al., Alzheimer's & Dementia, 7, 263-269, 2011). Patients who met the inclusion criteria for aMCI or mild dementia due to AD were invited for a screening initial visit at the Memory Evaluation and Therapeutic Service (METS) outpatient department at the Burke Rehabilitation Hospital. After informed consent was obtained from patients and their health care proxies, a physical examination including EKG, laboratory tests (Complete Blood Count, Complete Metabolic Panel, Vitamin B12, folate, Thyroid function tests), a neurological exam and the MMSE were administered. If eligible (Table 1), participants were referred to Westchester Imaging Center for an Amyloid PET/CT scan of the brain. Only participants with a positive amyloid scan were sent to CUIMC for a baseline F-18-FDG PET/CT scan of the brain. At the baseline visit, the cognitive tests were performed and blood drawn for measurement of thiamine, ThDP and ThMP by HPLC and APOE genotyping. Enrolled patients returned to the Burke outpatient clinic at month 3, 6, 9 and 12 for subsequent visits. At month 12, the final FDG/PET scan was performed at CUIMC.

The trial duration per participant was twelve months. Participants in the treatment group took one 300 mg capsule of benfotiamine in the morning and one in the evening. The participants in the placebo group took one 300 mg capsule in the morning and evening with microcrystalline cellulose without benfotiamine. At each visit, the patients returned the pill bottles for that period. The number of pills returned was used to assess compliance (the percent of pills consumed).

Characterization procedures. Amyloid-β was assessed using PET imaging with 18F-Betapir F18 PET [43] to help confirm the presence of AD pathology in study participants. Positivity was determined by a visual read. For APOE genotyping, total nucleic acid was isolated from whole blood samples using the MasterPure™ Complete DNA and RNA purification kit (Lucigen) with a starting volume of 150 μl of blood, according to the manufacturer's instructions. Genotyping of the two human APOE polymorphisms was carried out using the TaqMan® SNP genotyping assays (ThermoFisher Scientific): C_3084793_20 for SNP rs429358 and C_904973_10 for SNP rs7412. An initial 5 min step at 95° C. was followed by 40 cycles of 15 s at 95° C. and 30 s at 60° C. Genotyping was performed in duplicate with controls for all six possible APOE genotypes and no DNA controls using a QuantStudio™ 12K Flex real-time PCR system (ThermoFisher Scientific).

Treatment.

The trial duration per participant was twelve months. Participants in the treatment group took one 300 mg capsule of benfotiamine in the morning and one in the evening. The participants in the placebo group took one 300 mg capsule in the morning and evening with microcrystalline cellulose without benfotiamine. At each visit, the patients returned the pill bottles for that period. The number of pills returned was used to assess compliance (the percent of pills consumed). The benfotiamine and placebo were manufactured and provided by the Advanced Orthomolecular Research, Canada. They prepared the benfotiamine according to an FDA-approved IND, which was prepared by the Cornell Translational Science Center, and issued to the Burke Neurological Institute.

Cognitive Measures

The following cognitive tests were conducted at the intervals indicated in FIG. 1:

AD Assessment Scale-Cognitive Subscale (ADAS-Cog) was the primary outcome measure. It indicates the severity of the most important symptoms of Alzheimer's disease. It consists of 11 tasks measuring the disturbances of memory, language, praxis, attention and other cognitive abilities (S. Balsis et al., Alzheimer's & Dementia, 8, 288-294, 2012; G. Weyer et al., International Psychogeriatrics, 9, 123-138, 1997).

Clinical dementia rating (CDR) score. CDR is a five-point scale used to characterize six domains of cognitive and functional performance applicable to Alzheimer's disease and related dementias: Memory, Orientation, Judgment & Problem Solving, Community Affairs, Home & Hobbies, and Personal Care. A higher score indicates greater dementia (C. P. Hughes et al., British Journal of Psychiatry, 140, 566-572, 1982).

The Buschke Selective Reminding Test (SRT) is a standard diagnostic tool in the assessment of verbal memory. Several studies attest to its predictive value for dementia.

Neuropsychiatric Inventory (NPI). The NPI assesses a wide range of behaviors encountered in dementia patients to provide a means of distinguishing frequency and severity of behavioral changes. Ten behavioral and two neuro-vegetative domains are evaluated through an interview with the caregiver.

Alzheimer's Disease Cooperative Study-Activities of Daily Living (ADCS-ADL). The ADCS-ADLSIV is a caregiver-based ADL scale composed of 19 items developed for use in dementia clinical studies (D. Galasko et al., Alzheimer Disease and Associated Disorders, 11, S33-S39, 1997). It assesses the patient's performance of both basic and instrumental activities of daily living, such as those necessary for personal care, communicating and interacting with other people, maintaining a household, conducting hobbies and interests, as well as making judgments and decisions. Higher numbered scores and answers of “yes” reflect a more self-sufficient individual. Therefore, the higher total score correlates with higher cognitive function. The total score is the sum of all items and sub-questions.

Biomarker outcomes. AGE are formed during the Maillard reaction where reducing carbohydrates react with lysine side chains and N-terminal amino groups of various macromolecules, particularly proteins. AGE can adversely affect the function of these macromolecules. One of the most prevalent advanced glycation end products, N-epsilon-(carboxymethyl) lysine, has been implicated in oxidative stress and vascular damage. The quantity of AGE adduct in protein samples is determined by comparison with that of a known AGE-BSA standard curve. AGE levels were measured on plasma sample with a kit from ABCAM (AB238539), Cambridge, Mass., USA

Fluorodeoxyglucose (FDG) Positron Emission Tomography (PET)

Image acquisition, processing, and measurement. FDG PET imaging of glucose metabolism was acquired at baseline and after 12 months of treatment. All scans were acquired on a Siemens MCT 64 PET/CT PET-CT scanner at CUIMC. Study participants were maintained in an awake, at rest state with eyes and ears open in dim lighting during tracer uptake. Forty minutes after injection of the tracer, the emission image was acquired in four contiguous 5-minute frames. Frames were aligned with SPM 12, averaged, and then spatially normalized to the MNI template using SPM12 (https://www.fil.ion.ucl.ac.uk/spm/software/spm12/), resulting in one image per participant for each time point. Average voxel values within 90 regions of interest (ROIs) in the Automated Anatomic Labeling (AAL) Atlas were computed. A subset of 16 pre-specified bilateral regions of interest (ROIs) were chosen for the group analysis due to their relevance to AD including: posterior cingulate, precuneus, frontal, inferior parietal, mid temporal, hippocampus, paracentral lobule, and cerebellum. The paracentral lobule and cerebellum were included as reference regions given their relative preservation during AD progression.

Derivation of spatial covariance patterns for Glucose FDGPET.

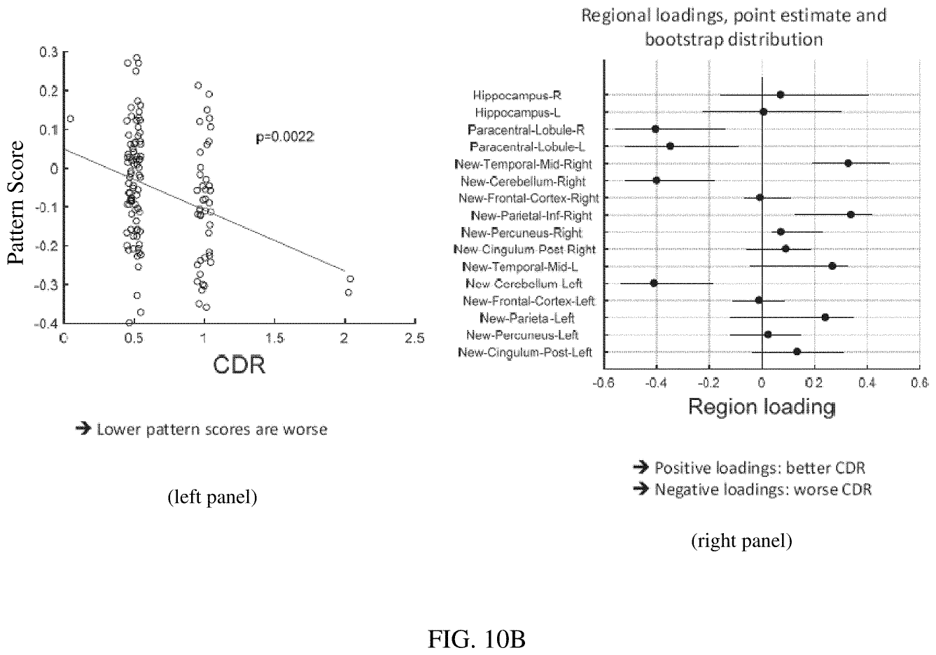

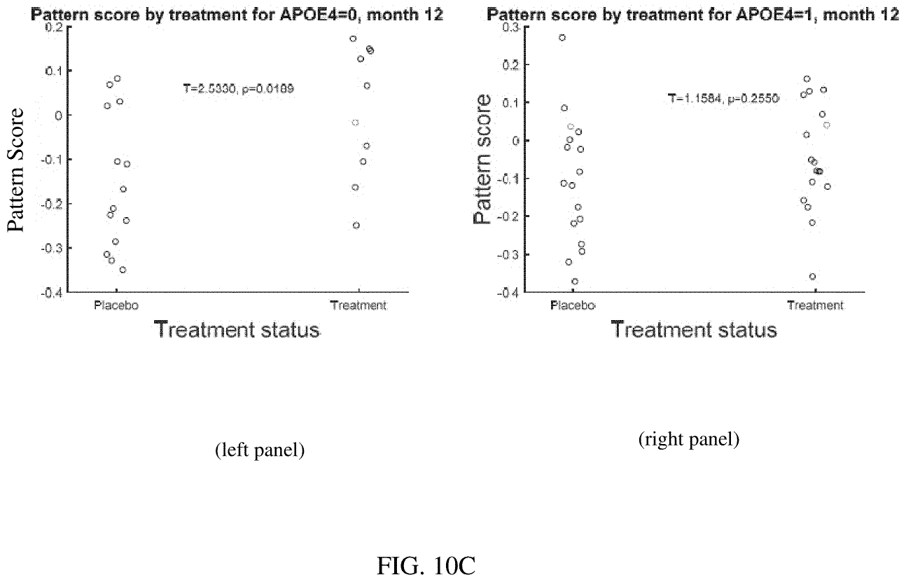

A multivariate machine learning approach was also applied to evaluate the FDG PET data (FIG. 10). Pattern-based methods have been increasingly applied to the evaluation of neurodegeneration and therapeutic response as they address the issue of complexity in comparing multiple regions and can increase signal to noise for analysis. Feature reduction was performed through use of the Scaled Subprofile Model (SSM), a form of Principal Components Analysis (PCA). The resulting components were used in regression modeling that determined spatial patterns of hypometabolism and hypermetabolism (or preservation relative to other regions) associated with the Clinical Dementia Rating (CDR) score.

Specifically, SSM by performing PCA on the PET-data array was run, with a subsequent brain-behavioral regression to derive a best-fitting pattern whose pattern scores correlates with the CDR score in a negative direction (i.e., the higher the pattern score, the lower the CDR). The best-fitting set of Principal Components was obtained via the Akaike criterion, and came out as PC1-2.

To help with the imputation of the multivariate analysis, a generic multivariate decomposition was written as: Y(s,x)=w(s) v(x)+ε(s,x), where Y denotes the (log-transformed) data which depends on a participant and time index s and the voxel location x. The pattern score w(s) is a scalar that solely depends on subject and time, but not voxel location, whereas the derived pattern v(x) depends on voxel location, but shows invariance across participants and time, i.e., does not depend on index s; ε(s,x) denotes residual signal that is dependent on participant, time and voxel location, but which was discarded for the present purposes. The pattern score w(s) was chosen to correlate negatively with CDR across the data. The pattern v(x) is normalized to have unity Euclidean norm, i.e. ||v||=1. This means that the pattern score carries all information about the strength of the signal associated with the spatial pattern. Higher values of w(s) imply higher values of pattern-associated FDGPET signal in direct proportion in all regions.

To estimate the topographic robustness of any patterns of interest, a bootstrap resampling procedure was performed 10,000 times, for which data were resampled with replacement and the complete analytic recipe was executed on the resampled data, generating distribution for pattern loadings. Regional loadings were considered robust if the 95% coverage interval (=[2.5%, 97.5%]) did not overlap with, and lay to one side of, zero. For the correct interpretation, it is worth noting that positive and negative loadings describe only relative, and not absolute differences, in the signal associated with any covariance pattern. Since the residual signal in ε(s,x) was stripped off, there cannot be assurance that there are absolute differences in the total data for the regions with robust loadings.

After deriving and estimating the topographic robustness of the pattern, the pattern score was inspected for an effect of treatment at baseline and follow-up, also broken down by APOE ε4 status.

Statistical Methods

The primary clinical outcome was ADAS-Cog, and secondary outcomes included the CDR score and fluorodeoxyglucose (FDGPET) imaging of the brain. AGE levels were an exploratory outcome. The primary analysis followed Intention-to-Treat (ITT) and the secondary analysis was per-protocol. The per-protocol analysis omitted one placebo participant who took benfotiamine from a commercial vendor. The ITT and per-protocol analysis are presented for the primary outcome ADAS-cog and the secondary measure CDR. For the other measures, only per-protocol analysis are presented.

Spearman correlation coefficient was used to assess the correlation between continuous variables. Student's t-test was used to compare the continuous variables between placebo and treatment groups, and Fisher's exact test was used to compare categorical variables between placebo and treatment groups. Specifically, two-sample Student's t-test was used to compare the score changes (ADAS score, normalized PET-related scores, etc.) from baseline between Placebo and Treatment groups when normality was satisfied, otherwise Wilcoxon Rank-sum test was used. ANCOVA was used to test the group difference while adjusting for covariates.

The primary analysis was done on the ITT data. The Last-Observation-Carry-Forward (LOCF) method was used to impute the missing values of ADAS total score and the secondary endpoints such as CDR as well for each time point. The primary analysis was done on ITT data which were imputed with LOCF method. Per-protocol analysis was done as a sensitivity analysis and as observational comparisons (F. J. Molnar et al., Canadian Medical Association Journal, 179, 751, 2008).

In the time to event analysis, time to ≥3 points of ADAS change was calculated based on whether the ADAS score changed from baseline ≥3 (event) at each time point. When no change ≥3 points was observed at any time point, the observation is censored and the last follow-up time (12 month) was used to calculate the duration. Kaplan-Meier estimator was then used to estimate probability of time-to-event. The difference between groups was tested by log-rank test for statistical significance.

As sensitivity analyses, repeated-measure ANOVA, generalized estimating equation (GEE) and Mixed effect model and Wilcoxon Rank-sum test were also performed on primary endpoints with and without imputation to compare differences between placebo and treatment groups.

Subgroup analyses in MMSE, APOE and sex were either in the per-protocol analysis or exploratory. A Student's t-test was used in each of the subgroup comparisons. An ANCOVA was also used to analyze the treatment difference while adjusting for each of these covariates. Interaction between MMSE and ADAS-cog responses was assessed by ANCOVA with interaction term.

All statistical tests were two-sided with an alpha level of 0.05 as the significance cutoff. All analyses were performed in statistical software SAS Version 9.4 (SAS Institute, Cary, N.C.).

Results

Characteristics of the populations at baseline.

Pre-screening of 634 patients at the METS at the Burke Rehabilitation Hospital excluded all but 120 participants (FIG. 1). Only eighty-three of these patients were amyloid positive. Twelve declined to participate. Seventy-one of these participants agreed to be part of the trial, and were randomized to receive either placebo or benfotiamine. Eight subjects were prematurely discontinued from the trial prior to Month 12. Three participants were withdrawn due to non-compliance <80%; three withdrew consent due to unwillingness to complete study procedures; one participant was lost to follow-up and one was withdrawn by PI due to physical limitations. None of the participants randomized to the treatment group withdrew due to adverse reactions or adverse effects. Since the ones who withdrew did not have final scores, their dropout did not affect 12 month scores. After the trial completion and after the data were locked, one patient in the placebo group was determined to be on benfotiamine from another source and was excluded from the per-protocol analysis. Thus, 37 (placebo) and 34 (benfotiamine) were included in the ITT analysis, and 36 (placebo) and 34 (benfotiamine) were included in the per-protocol analysis.

Whether the patient took the required medication was referred to as compliance. If the patients who withdrew are included, the percent compliance in the placebo group was 87.7 (3.5)% and in the treatment group was 89.8 (3%). If the patients that withdrew are not included, the percent compliance in the placebo group was 94.1 (1.3)% and in the treatment groups percent compliance was 94.8 (1.4)%.

Tables 2A-2D, below, provide baseline comparisons between benfotiamine (n=34) and placebo (n=36). The demographic characteristics of the patients are described in Table 2A below. The randomization procedure was based on the order of patient entry into the study. There were no statistically significant differences in age, race, MMSE, and demographic or clinical characteristics. The goal to recruit patients with an average MMSE of 26 was met. The percentage of females in the benfotiamine group (67.6%) was higher than in the placebo group (50%). Although the distribution by race was similar, only 2.9% of the population was Non-Hispanic Black. The distribution of APOE ε4 carriers and non-carriers (60% and 40%, respectively) in the whole population was reflected in the benfotiamine (64.7 and 35.3%, respectively) and placebo (55.6% and 44.4%, respectively) groups. Nearly identical proportions were also observed for males (58.6% and 41.4%) and females (61% and 39%). The scores on the neuropsychological tests at baseline did not differ between the two groups, with the exception of NPI, which differed between groups at baseline (p=0.040) (Table 2B below).

TABLE 2A
Baseline demographic characteristics

| | Total | Placebo | Benfotiamine | P value |
|---|---|---|---|---|
| Age | | | | |
| Mean (SD) | 75.77 (7.01) | 75.81 (7.19) | 75.74 (6.91) | 0.967 T |
| Gender | | | | |
| Female | 41 (58.6) | 18 (50.0) | 23 (67.6) | 0.153 F |
| Male | 29 (41.4) | 18 (50.0) | 11 (32.4) | |
| Race | | | | |
| Black | 2 (2.9) | 1 (2.8) | 1 (2.9) | 1.000 F |
| White | 68 (97.1) | 35 (97.2) | 33 (97.1) | |
| Ethnicity | | | | |
| Hispanic/Latino | 4 (5.7) | 4 (11.1) | 0 (0.0) | 0.115 F |
| Not Hispanic/Latino | 66 (94.3) | 32 (88.9) | 34 (100) | |
| MMSE total | | | | |
| Mean (SD) | 25.33 (2.63) | 25.33 (2.52) | 25.32 (2.78) | 0.988 T |
| Dichotomized MMSE | | | | |
| <26 | 34 (48.6) | 18 (50.0) | 16 (47.1) | 0.816 F |
| >=26 | 36 (51.4) | 18 (50.0) | 18 (52.9) | |
| APOE genotype | | | | |
| ‘2, 3’ | 4 (5.7) | 2 (5.6) | 2 (5.9) | 0.883 F |
| ‘2, 4’ | 1 (1.4) | 0 (0.0) | 1 (2.9) | |
| ‘3, 3’ | 24 (34.3) | 14 (38.9) | 10 (29.4) | |
| ‘3, 4’ | 34 (48.6) | 17 (47.2) | 17 (50.0) | |
| ‘4, 4’ | 7 (10.0) | 3 (8.3) | 4 (11.8) | |

T: t-test (with equal variances); “Fisher's exact t” test

TABLE 2B
Baseline neuropsychological outcome measures

| | Total | Placebo | Benfotiamine | P value |
|---|---|---|---|---|
| ADAS total score | 15.34 (6.36) | 15.50 (6.61) | 15.19 (6.16) | 0.835 t |
| (ITT) | | | | |
| ADAS total score | 15.34 (6.40) | 15.48 (6.70) | 15.19 (6.16) | 0.849 t |
| (Per protocol) | | | | |
| CDR score | 0.50 (0.50-1.00) | 0.50 (0.50-1.00) | 0.50 (0.50-1.00) | 0.334 w |
| Median (range) | | | | |
| ADCS-ADL total score | 47.44 (4.29) | 47.42 (4.65) | 47.47 (3.95) | 0.959 t |
| NPI | 13.50 (10.44) | 11.03 (10.15) | 16.12 (10.23) | 0.040 t |
| Buschke score | 27.09 (9.74) | 26.03 (9.01) | 28.21 (10.49) | 0.354 t |

Values are Mean (SD).
t denotes t-test (with equal variances).
w Wilcoxon rank sum test

TABLE 2C
Baseline thiamine, ThDP, and ThMP

| | Total | Placebo | Benfotiamine | P Value |
|---|---|---|---|---|
| Thiamine | 5.72 (11.31) | 5.26 (4.50) | 6.20 (15.56) | 0.735 |
| Mean (SD) | | | | |
| Thiamine | 69.71 (19.40) | 74.46 (20.21) | 64.82 (17.50) | 0.038 |
| diphosphate | | | | |
| Mean (SD) | | | | |
| Thiamine | 3.21 (1.72) | 3.46 (1.83) | 2.97 (1.59) | 0.250 |
| monophosphate | | | | |
| Mean (SD) | | | | |

Comparisons were by t-test (with equal variances)

TABLE 2D
Baseline comparison of FDGPET

| | Total | Placebo | Benfotiamine | P value |
|---|---|---|---|---|
| Posterior cingulate | | | | |
| Left | 0.85 (0.09) | 0.83 (0.10) | 0.87 (0.08) | 0.087 |
| Right | 0.81 (0.07) | 0.79 (0.07) | 0.82 (0.06) | 0.069 |
| Percuneus | | | | |
| Left | 1.09 (0.09) | 1.08 (0.10) | 1.10 (0.08) | 0.332 |
| Right | 1.09 (0.10) | 1.08 (0.11) | 1.11 (0.09) | 0.287 |
| Medial temporal | | | | |
| Left | 0.97 (0.11) | 0.94 (0.11) | 1.00 (0.10) | 0.022 |
| Right | 1.01 (0.12) | 0.98 (0.12) | 1.04 (0.10) | 0.020 |
| Frontal cortex | | | | |
| Left | 0.99 (0.09) | 0.98 (0.09) | 0.99 (0.09) | 0.480 |
| Right | 1.01 (0.09) | 1.00 (0.09) | 1.03 (0.08) | 0.268 |
| Hippocampus | | | | |
| Left | 0.74 (0.08) | 0.74 (0.08) | 0.75 (0.08) | 0.938 |
| Right | 0.76 (0.09) | 0.75 (0.10) | 0.76 (0.08) | 0.764 |
| Entorhinal_cortex | | | | |
| Left | 0.88 (0.13) | 0.88 (0.14) | 0.87 (0.13) | 0.693 |
| Right | 0.89 (0.18) | 0.87 (0.21) | 0.90 (0.14) | 0.477 |
| Prefrontal_cortex | | | | |
| Left | 0.82 (0.09) | 0.81 (0.09) | 0.83 (0.08) | 0.417 |
| Right | 0.87 (0.09) | 0.86 (0.10) | 0.88 (0.08) | 0.293 |
| Whole brain | 0.88 (0.05) | 0.87 (0.06) | 0.89 (0.05) | 0.122 |

All values were normalized to the cerebellum as described in methods. All values are mean (SD). All comparison were by the t-test (equal variances).

Baseline thiamine and ThMP, but not ThDP distributions were similar in the two groups. Blood thiamine diphosphate (ThDP) was lower (p=0.038) in the benfotiamine group (Table 2C). In agreement with the literature (X. Pan et al., EBioMedicine, 3, 155-162, 2016), ThDP was lower in females than males (p=0.0003). At baseline, ThDP did not correlate with MMSE (p=0.644), CDR (p=0.618), ADAS-cog (p=0.883) or whole brain glucose utilization (p=0.644).

Baseline FDGPET measures are presented in Table 2D. In agreement with prior findings, FDG PET in whole brain at baseline correlated with the MMSE (Spearman correlation, r=0.288, p=0.015). Brain glucose utilization was 4.4% higher in females than males (p=0.003). At baseline, FDG PET in the mid-temporal region was significantly higher in the benfotiamine treatment group than placebo group (p=0.020), and the cingulate was higher in the treatment group at trend level (p=0.069) (Table 2D).

Safety profile. Tables 3A-3D, below, show the consequences of a twelve-month treatment with benfotiamine. No adverse events related to the 2×300 mg benfotiamine per day were observed and patients did not complain about the medication (Table 3A below).

TABLE 3A
Data showing that benfotiamine did not cause adverse events

| SYMPTOM | PLACEBO (n = 36) | TREATMENT (n = 34) |
|---|---|---|
| Anxiety | 4 (11%) | 5 (14%) |
| Bruise | 5 (14%) | 2 (6%) |
| Cold symptoms | 3 (8%) | 3 (8%) |
| Depression | 2 (6%) | 1 (3%) |
| Dizziness | 3 (8%) | 3 (8%) |
| Fall | 12 (34%) | 6 (17%) |
| Head injury | 2 (6%) | 0 (0%) |
| Heart arrhythmia | 2 (6%) | 1 (3%) |
| Pain | 4 (11%) | 5 (14%) |
| Pneumonia | 3 (8%) | 0 (0%) |
| Sprain | 2 (6%) | 0 (0%) |
| Surgery | 3 (8%) | 1 (3%) |
| Allergy | 2 (2%) | 0 (0%) |
| Gastrointestinal problem | 12 (34%) | 9 (26%) |
| Stroke | 0 (0%) | 2 (6%) |
| Total | 59 | 38 |

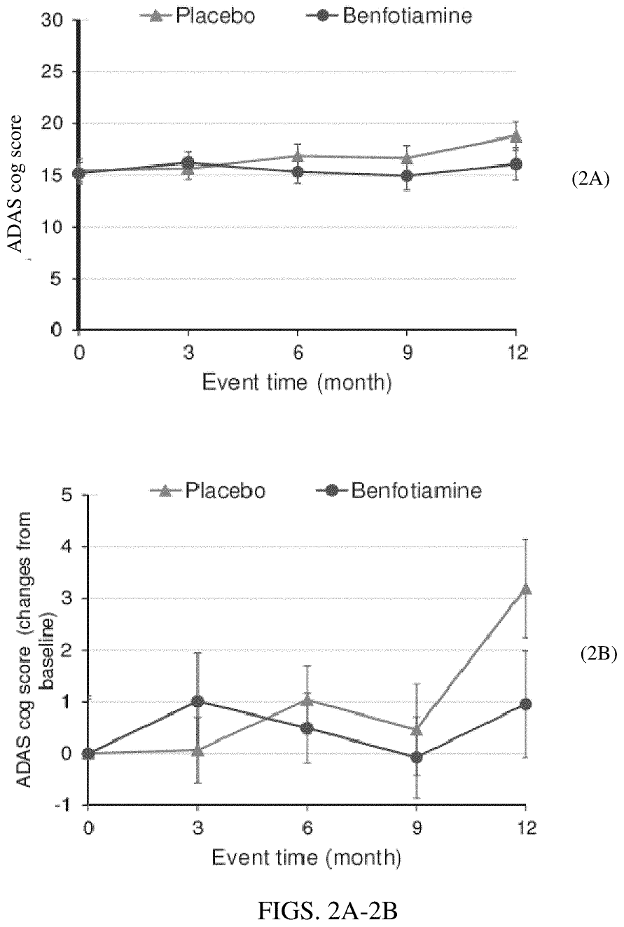

Benfotiamine and ADAS-Cog changes (FIGS. 2A, 2B, Table 3B).

A comparison of unadjusted changes from baseline to 12 months with ITT analysis revealed a difference between the benfotiamine and placebo groups favoring benfotiamine using a mixed effect model (p=0.071), GEE (p=0.137), and a non-parametric Wilcoxon rank sum test (p=0.098) (FIG. 2, Table 3B). At 12 months, the change in the placebo group was 3.26 whereas in the benfotiamine group the change was 1.39. This difference was not apparent at 3, 6, or 9 months. The per-protocol analysis (Table 3B) suggested that the differences were significant when analyzed by a mixed effect model (p=0.035), GEE (p=0.069) or Wilcoxon Rank-Sum (p=0.049). The sub-category exploratory analysis of ADAS-cog revealed that the changes from baseline in the commands component (p=0.001) and the word finding difficulty (p=0.033) were significant at 12 months.

TABLE 3B
Unadjusted comparison of the baseline to month 12 in ADAS
score between benfotiamine and control (Per-protocol)

| Variable | Total | Placebo | Benfotiamine | P¹ |
|---|---|---|---|---|
| ADAS score change | 2.10 (5.59) | 3.2 (5.66) | 0.96 (5.41) | 0.125 ^{[T]} |
| Mean (SD) | | | | |

Repeated measures ANOVA P-value: 0.355
Mixed effect model P-value: 0.056
GEE P-value: 0.107
Wilcoxon Rank sum P-value: 0.069
¹P values obtained from the statistical tests:
^{[T]} - T-test (equal variances);

An exploratory analysis of effect modification by sex suggests that males might have been more responsive to benfotiamine, although none of the differences were statistically significant. Furthermore, there was no effect modification by APOE ε4 allele carrier status. Finally, no significant correlation occurred between blood thiamine, ThDP or ThMP values and ADAS-cog. No significant interaction was found between MMSE score and ADAS-cog response (p=0.122), but a post-hoc analysis suggests that benfotiamine had a stronger response among those with a higher MMSE at baseline (MMSE >=26 difference in change ADAS-cog was significant (p=0.027)) whereas this was not the case for MMSE<26 (p=0.99).

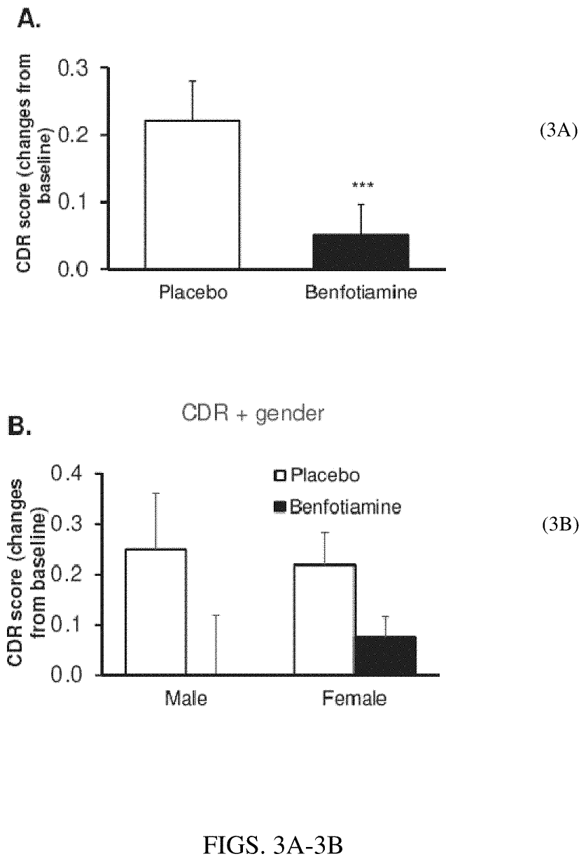

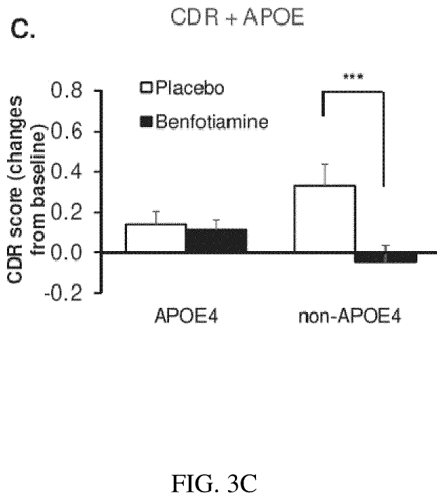

CDR. Mean change in global CDR from baseline to 12 months was significantly different between placebo and benfotiamine groups (p=0.034), favoring the benfotiamine group (FIGS. 3A-3C). The difference in the placebo group was 0.22 whereas the change in the benfotiamine group was 0.05, corresponding to a reduction of deterioration by 77%. The mean change in CDR-SB from baseline to 12 months showed a difference at trend level between placebo and benfotiamine groups (p=0.078). In an analysis of individual CDR subscores, the “home and hobbies score” differed between groups (p=0.032) whereas other subscores did not differ.

APOE ε4 status (FIG. 3C), but not sex (FIG. 3B), was associated with a differential response to benfotiamine. The performance of males and females was not significantly different (FIG. 3B). The change from baseline in females 0.219 was nearly identical to that in males. However, the non-APOE ε4 group seemed to respond much more than those with the ε4 allele (FIG. 3C). Indeed, the change from baseline was significant in the non-APOE ε4 group (p=0.013) although only eleven participants were in this category. No significant interaction was found by comparing patients that had MMSE values >=26 vs<26 (p=0.878).

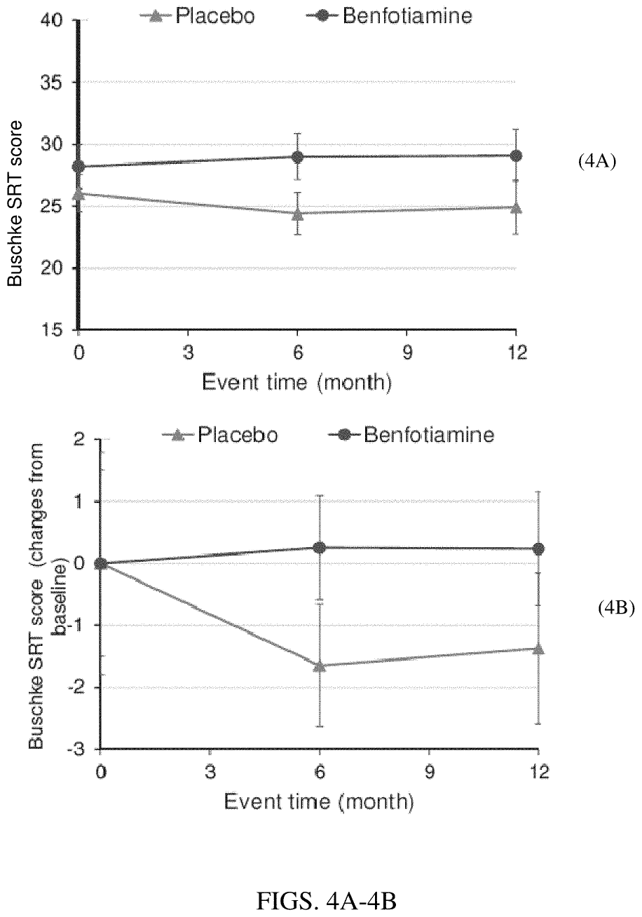

The Buschke SRT is plotted in FIGS. 4A and 4B. As shown, no significant change in the SRT (p=0.177) or change in score (0.315) occurred. Placebo treated participants showed a downward trend while benfotiamine treated participants had stable scores. Trend analysis shows that the non-APOE ε4 are the most responsive at 6 months (compared baseline p=0.028) and 12 months (compared to baseline p=0.066).

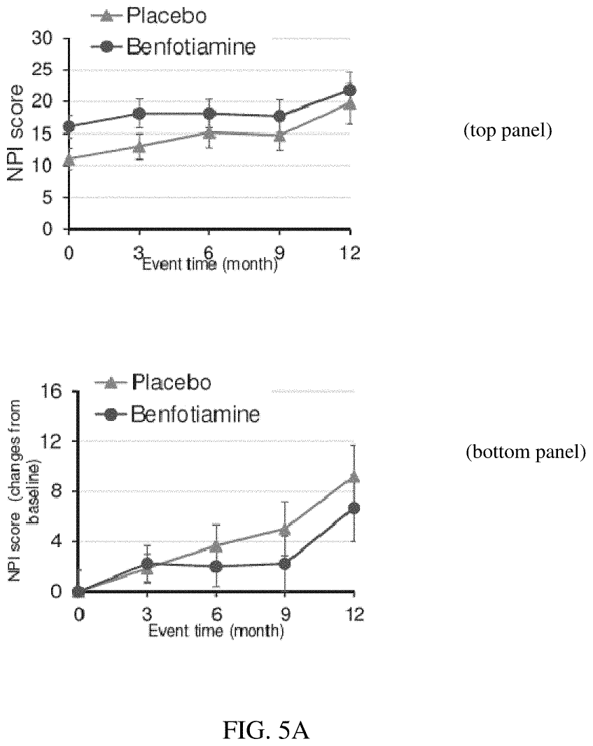

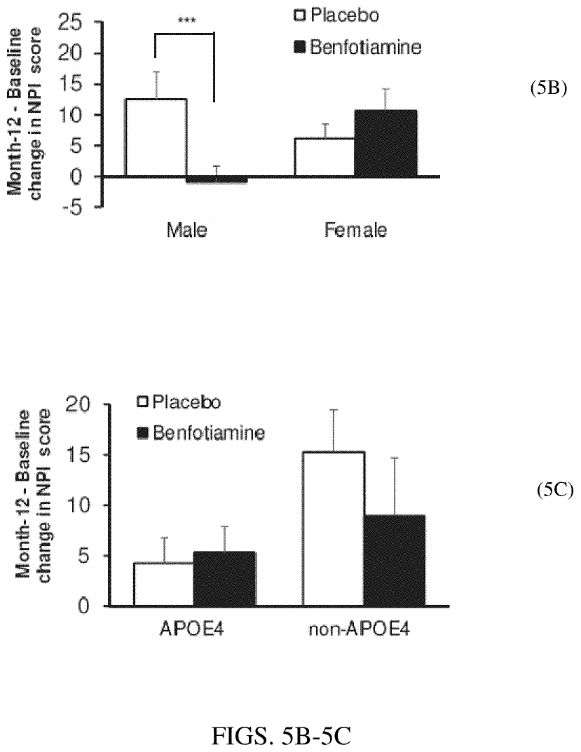

The NPI score is plotted in FIGS. 5A-5C. No differences in change in NPI were observed with benfotiamine treatment when the whole population was analyzed (FIGS. 5A, top and bottom panels). However, as shown in FIG. 5B, benfotiamine was associated with significantly reduced scores in males at month 9 (0.014) and month 12 (p=0.035). As shown in FIG. 5C, the effects of benfotiamine were not altered by APOE4 status.

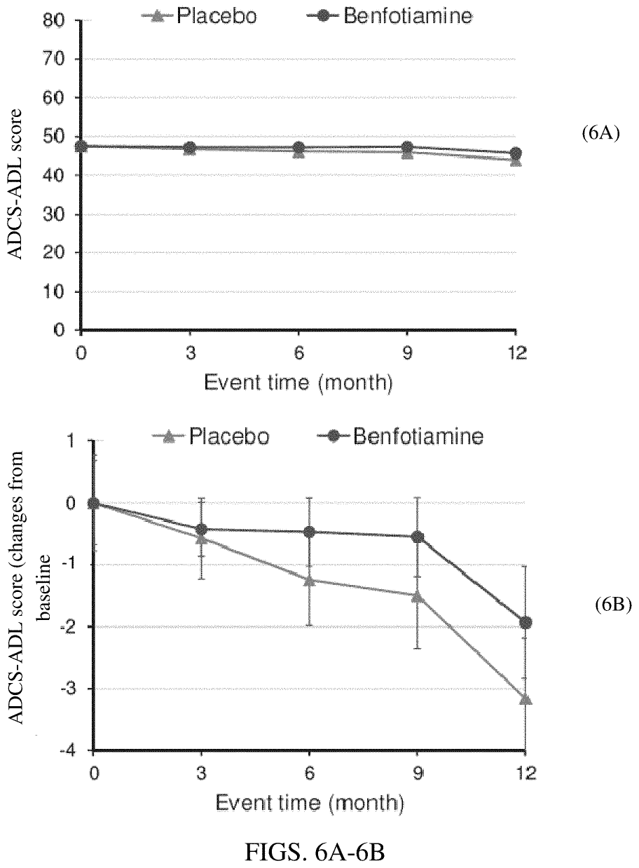

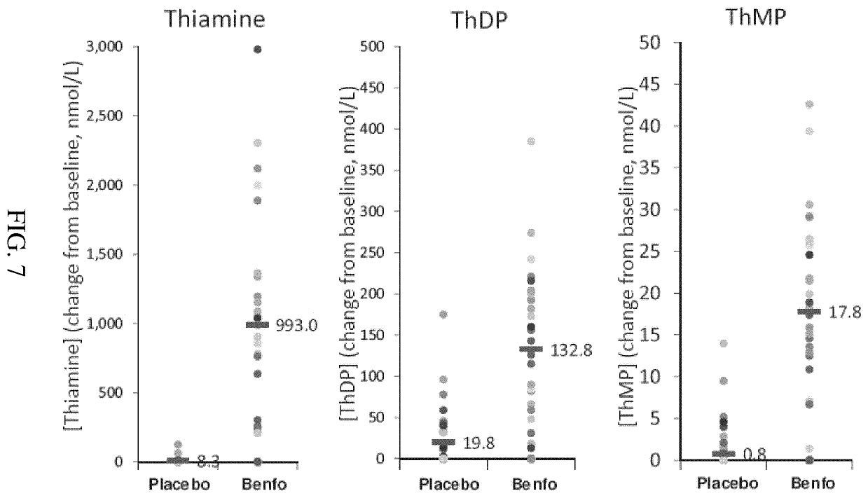

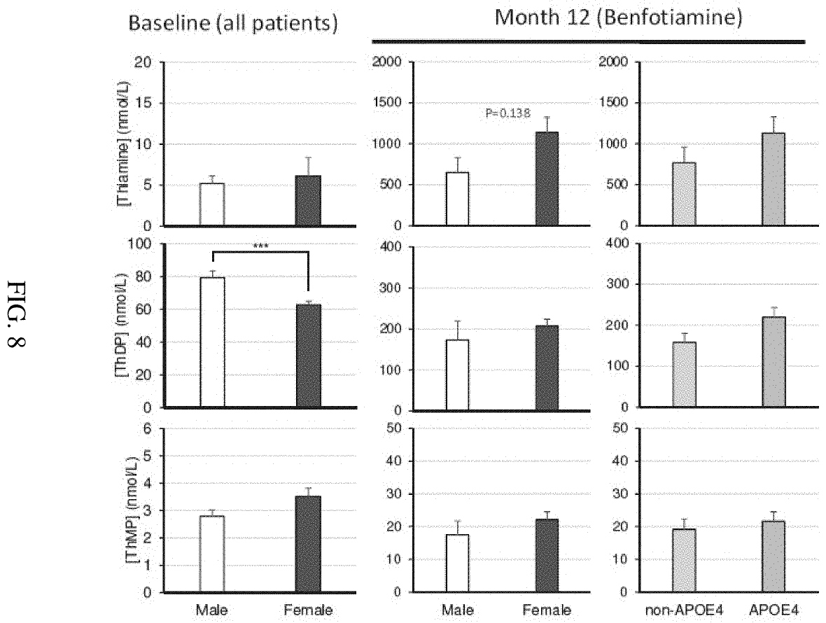

The ADCS-ADL score is plotted in FIGS. 6A and 6B. As shown, no significant differences were observed in ADCS-ADL. In the sub-analysis of sex and APOE, a trend was observed that was consistent with a beneficial effect of benfotiamine.

FIGS. 7 and 8 show the response of thiamine, ThDP, and ThMP to benfotiamine treatment (see also Table 3C below).

TABLE 3C
Changes in Thiamine, ThDP and ThMP after 12 months of placebo
(n = 36) or benfotiamine (n = 34).

| | | |
|---|---|---|
| Baseline | 12 months | P value |

Changes in thiamine and its esters after 12 months of placebo

| | | | |
|---|---|---|---|
| Thiamine | 5.48 ± 0.77 | 13.64 ± 4.06 | 0.044 |
| Thiamine diphosphate | 74.46 ± 3.42 | 91.70 ± 7.94 | 0.044 |
| Thiamine monophosphate | 3.38 ± 0.31 | 4.05 ± 0.73 | 0.382 |

Changes in thiamine and its esters after 12 months of benfotiamine

| | | | |
|---|---|---|---|
| Thiamine | 6.20 ± 2.67 | 999.51 ± 147.4 | <0.001 |
| Thiamine diphosphate | 64.82 ± 3.00 | 197.39 ± 17.75 | <0.001 |
| Thiamine monophosphate | 2.97 ± 0.27 | 20.73 ± 2.16 | <0.001 |

The 161 fold increase in in blood thiamine indicated the administration of the drug was successful. In the placebo group, small increases for the levels of thiamine (5.5 to 13.6; p=0.044) and ThDP (74.5 to 91.7; p=0.044) occurred, but not ThMP (3.4 to 4.0; p=0.382) (Table 3C). After completion of the trial, it was discovered that one patient in the placebo group took commercial benfotiamine during the trial. Consequentially, data from the patient was excluded for all per-protocol analysis. The twelve-month treatment with benfotiamine significantly elevated blood thiamine from 6.2 to 999 (161 fold) above baseline, ThDP (two fold) and ThMP (five fold) (Table 3C). Although the differences were significant, the scatter grams revealed large variations (FIG. 7). These changes were apparent even though the timing between taking the last capsule and taking blood were not standardized. The much larger changes than expected may be related to the duration of the treatment or the purity of the benfotiamine.

There was a trend for APOE ε4 and sex related differences in thiamine response to benfotiamine, but the differences were not significant (FIG. 8). Thiamine levels after benfotiamine were about two times higher in females than males. Thiamine values were approximately 50% higher in APOE ε4 carriers than non-APOE ε4 carriers.

The concentrations of blood thiamine, ThDP, ThMP after benfotiamine treatment did not correlate with ADAS-cog scores (p=0.736, 0.917, 0.500, respectively) nor CDR (p=0.762, 0.896, 0.767, respectively).

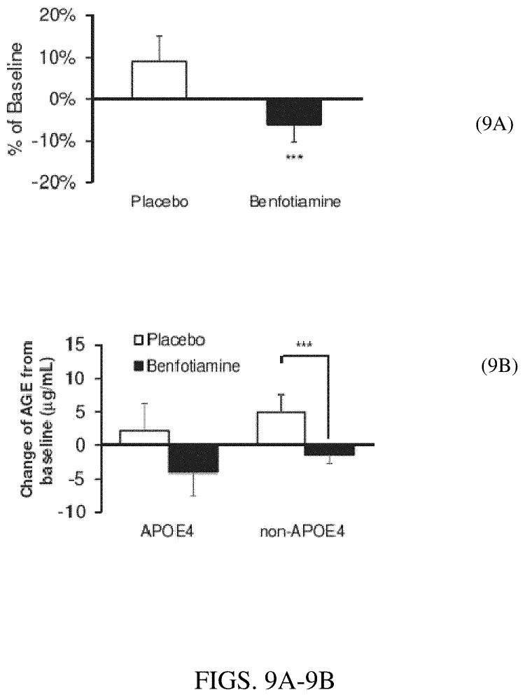

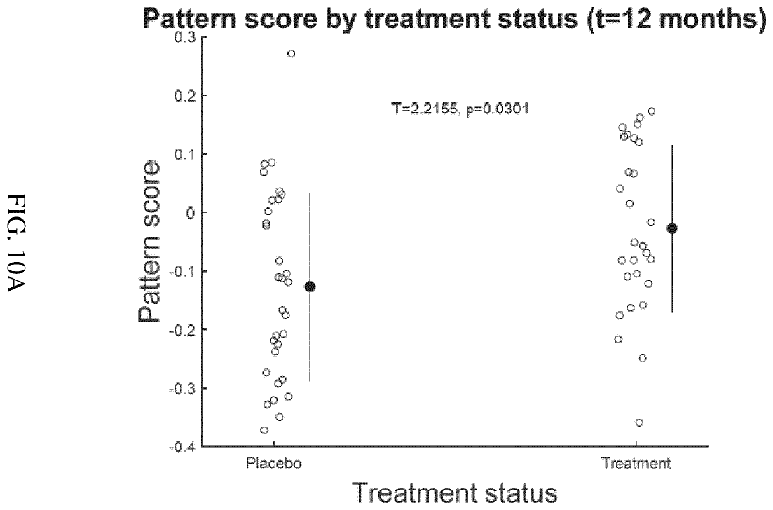

The response of advanced glycation end products (AGE) to benfotiamine treatment is shown in FIGS. 9A and 9B. As shown, benfotiamine inhibited the increase in AGE over the course of the disease and the effect was more apparent in non-APOE ε4 patients.

The response of FDGPET to benfotiamine treatment

The comparison of regions of interest is presented in Table 3D (below), using the paracentral lobule and cerebellum as the reference region. No significant differences were observed between the benfotiamine and placebo populations in the pre-specified regions of interest.

TABLE 3D
Comparison of the Month_12 - Baseline change in FDGPET between
benfotiamine (n = 34) and placebo (n = 36)

| | Total | Placebo | Benfotiamine | P value |
|---|---|---|---|---|
| Posterior cingulate | | | | |
| Left | −0.03 (0.03) | −0.03 (0.04) | −0.02 (0.03) | 0.629 |
| Right | −0.02 (0.03) | −0.02 (0.03) | −0.02 (0.03) | 0.742 |
| Parietal | | | | |
| Left | −0.02 (0.04) | −0.03 (0.04) | −0.02 (0.05) | 0.448 |
| Right | −0.03 (0.04) | −0.03 (0.04) | −0.02 (0.04) | 0.323 |
| Percuneus | | | | |
| Left | −0.03 (0.04) | −0.02 (0.04) | −0.03 (0.04) | 0.719 |
| Right | −0.03 (0.04) | −0.03 (0.04) | −0.03 (0.04) | 0.722 |
| Medial temporal | | | | |
| Left | −0.03 (0.04) | −0.03 (0.04) | −0.03 (0.04) | 0.956 |
| Right | −0.03 (0.05) | −0.03 (0.05) | −0.03 (0.05) | 0.748 |
| Frontal cortex | | | | |
| Left | −0.02 (0.04) | −0.02 (0.04) | −0.03 (0.04) | 0.646 |
| Right | −0.02 (0.04) | −0.02 (0.04) | −0.03 (0.04) | 0.616 |
| Hippocampus | | | | |
| Left | −0.02 (0.04) | −0.02 (0.05) | −0.01 (0.04) | 0.451 |
| Right | −0.02 (0.04) | −0.02 (0.05) | −0.02 (0.04) | 0.503 |
| Entorhinal_cortex | | | | |
| Left | −0.02 (0.11) | −0.01 (0.10) | −0.02 (0.11) | 0.774 |
| Right | −0.02 (0.09) | −0.01 (0.08) | −0.02 (0.10) | 0.502 |
| Prefrontal_cortex | | | | |
| Left | −0.02 (0.04) | −0.02 (0.04) | −0.02 (0.04) | 0.742 |
| Right | −0.02 (0.04) | −0.02 (0.04) | −0.03 (0.04) | 0.559 |
| Whole_brain | −0.01 (0.02) | −0.02 (0.02) | −0.01 (0.02) | 0.753 |

The data in FIG. 10B shows that the multivariate pattern derived through the regression against CDR correlated negatively with CDR (p=0.002) across all participants and time points. Robust positive loadings, i.e., with more than 97.5% of bootstrap loadings larger than zero, were found in the right precuneus, inferior parietal and mid frontal cortex: higher relative signal in these areas was associated with a better (=lower) CDR score. Robust negative loadings, i.e., with more than 97.5% of bootstrap loadings smaller than zero, were found in the bilateral paracentral lobules and bilateral cerebellum: higher relative signal at these locations was associated with a higher (=worse) CDR score. As shown in FIG. 10A, pattern scores showed a significantly higher change from baseline to 12 months in treated than untreated participants. However, a difference was observed between placebo and treatment arm at baseline (T=2.1582, p=0.034) when APOE status was not considered. Calculation of differences with sex was complicated by differences in the rates of the two groups at baseline.

Stratification by APOE ε4 revealed that that the CDR-derived FDG PET pattern showed a treatment effect at 12 months in APOE ε4 negative population (p=0.019) but not in APOE ε4 positive population (p=0.255) (FIG. 10C). In the APOE positive population there was no difference between treatment groups at baseline (p=0.164); in the APOE negative population, pattern scores were higher at trend level (p=0.086) in the benfotiamine group.

For 59 participants who completed follow-up, the longitudinal change in pattern score (follow-up minus baseline) also correlated negatively with the accompanying change in CDR score (R=−0.446, p<0.001). No difference in longitudinal change was observed between treated and untreated participants (p=0.638). Additional analyses to adjust for any baseline differences and to explore other baseline heterogeneity effects or comparison patterns were deferred for subsequent evaluation.

### DISCUSSION

The results show that benfotiamine administration in patients with aMCI and dementia due to AD is safe and successful in increasing peripheral thiamine levels. The trial provides preliminary evidence of efficacy of benfotiamine on cognitive and functional outcomes. In aggregate, the present results provide proof of principle that justify testing the efficacy of benfotiamine in ameliorating cognitive and functional decline among participants with aMCI and dementia due to AD in a trial with a larger sample size and study duration. Measures of blood thiamine (a pharmacokinetic marker of drug delivery), FDGPET patterning (a CNS biomarker of synaptic activity) and serum AGE (a peripheral biomarker of metabolic dysregulation) provided further evidence of the effects of benfotiamine that could benefit cognition.

The large increases in whole blood thiamine, ThDP, and ThMP provide a robust indication that oral tablets effectively delivered the treatment. Indeed, the 161-fold increase serum thiamine was more robust than predicted, but the variation was large. Appreciable differences in thiamine levels were observed by sex (two-fold) and APOE-epsilon4 carrier status (three-fold) following treatment, but these observations need to be replicated in a larger sample.

The ability of blood thiamine or its esters to predict AD at baseline that was suggested by other trials was not evident in patients of the present study. Unlike previous studies which included more severe patients, blood ThDP did not correlate significantly with MMSE (0.664), CDR (0.618) or ADAS-cog (0.883) at baseline or following benfotiamine. Thus, the baseline studies are not supportive of a critical role of blood ThDP in AD. The present studies do support the finding that ThDP is lower in females than males. These results indicate that thiamine, ThDP and ThMP should be tested in a subsequent study, and additional aspects of thiamine homeostasis such as cellular localization or ThDP effect on transketolase should be tested as well. At minimum, the blood measures provide a measure of drug delivery.

The significant correlation of MMSE and the normalized FDG PET at screening, as well as the correlation between CDR and the derived multivariate pattern, are consistent with the well-documented tight relation of glucose metabolism to AD. Several factors may have contributed to the lack of FDG PET treatment effect findings despite the large measured changes in blood thiamine and observed differences in ADAS-cog changes. These include baseline heterogeneity in regional hypometabolism, the number of participants having both initial and post-treatment scans, use of CDR as the sole target outcome for the progression pattern, and the very small longitudinal changes that occur in FDG PET over 12 months in this mild population. Next steps include alternate apriori and data driven pattern-based analyses to further understand these relationships. As other potential considerations, the positive effects of benfotiamine/thiamine, including improved cognition, in neurodegeneration occur with minimal change in ThDP (J. Vignisse et al., Molecular and Cellular Neuroscience, 82, 126-136, 2017). Thus, benfotiamine/thiamine could be acting at steps of glucose metabolism that do not change brain glucose uptake or by one of thiamine/benfotiamine's actions not directly linked to metabolism. Thiamine also regulates activities of enzyme like malate dehydrogenase and glutamate dehydrogenase (O. A. Mezhenska et al., Biochemistry (Moscow), 85, 27-39, 2020). Thiamine is known to act as an antioxidant and may act directly in cholinergic transmission. Thiamine serves as an allosteric regulator of many proteins. Benfotiamine and thiamin may act as Nrf2 activators, which would help the brain deal with many oxidative insults. Finally, benfotiamine/thiamine may be acting on endothelial cells as has been demonstrated in studies of diabetes.

The CDR, FDGPET data, and AGE response to benfotiamine suggest that AD patients without APOE ε4 were more responsive to benfotiamine in this study population. The diminished response did not seem to be a difference in drug availability since blood thiamine (+46%), and its esters were all higher in patients with APOE ε4 following benfotiamine (not statistically significant). Patients with APOE ε4 may have a more severe form of the disease since they have more plaques and they occur earlier (W. J. Jansen et al., JAMA, 313, 1924-1938, 2015). APOE ε4 carriers have higher levels of the glyoxal, fluorescent AGEs, Nε-carboxymethyllysine, and the receptor for AGE (sRAGE) (p=0.018) when compared to non-carriers (P. Deo et al., The Journals of Gerontology. Series A, Biological Sciences and Medical Sciences, glz259, 2019).

The role of AGE in AD as a biomarker and progression of disease is not well developed. Recent studies demonstrate that the development of AGE parallels the development of the cognitive deficit (P.-S. Chou et al., Journal of Alzheimer 's Disease, 72, 191-197, 2019). The AGE pentosidine known to be an indicator of Alzheimer's disease. Methylglyoxal and glyoxal levels in serum are higher in MCI patients. Methylglyoxal in serum distinguishes MCI from controls but not from AD. Meanwhile, serum glyoxal levels differentiate MCI from control and AD groups. The levels of carboxymethylysine in serum correlate negatively with the clinical cognitive as measured by MMSE (M. Haddad et al., Journal of Alzheimer's Disease, 69, 751-762, 2019). AGE increase in healthy APOE ε4 and this may provide a link between APOE ε4 and AGE and the present responses. Both sex and APOE status are known to alter the AD serum metabolome. In animals, even mild thiamine deficiency leads to increases in AGE (F. Depeint et al., Nutrition Research, 27, 698-704, 2007).

While there have been shown and described what are at present considered the preferred embodiments of the invention, those skilled in the art may make various changes and modifications which remain within the scope of the invention defined by the appended claims.

What is claimed is:

1. A method for treating a subject having or likely to develop a neurodegenerative disease, the method comprising administering to the subject a pharmaceutically effective amount of a compound within the following generic structure: 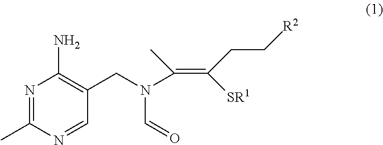
wherein R¹ is either —R or —SR, wherein R is a hydrocarbon group containing 1-20 carbon atoms and optionally containing one or more heteroatoms selected from oxygen, nitrogen, and sulfur; and R² is selected from the group consisting of —OR′, —OPO₃²⁻, and —OC(O)R′, wherein R′ is a hydrogen atom or a hydrocarbon group containing 1-6 carbon atoms;
and wherein Formula (1) further comprises pharmaceutically acceptable salts, solvates, and polymorphs thereof.

2. The method of claim 1, wherein the neurodegenerative disease is characterized by amyloid plaques and/or neurofibrillary tangles.

3. The method of claim 1, wherein the subject is positive for one or more markers selected from the group consisting of amyloid plaques in the brain, neurofibrillary tangles in the brain, decline in brain glucose metabolism, decline in thiamine diphosphate-dependent enzyme activity in the blood or brain, and increase in advanced glycation end products in the blood, urine, or brain.

4. The method according to any one of claims 1-3, wherein the neurodegenerative disease is Alzheimer's Disease.

5. The method according to any one of claims 1-4, wherein the neurodegenerative disease is characterized by mild cognitive impairment.

6. The method of claim 5, wherein the mild cognitive impairment is mild dementia.

7. The method according to any one of claims 1-6, wherein the subject is administered at least 300 mg of a compound of Formula (1) per day.

8. The method according to any one of claims 1-6, wherein the subject is administered at least 600 mg of a compound of Formula (1) per day.

9. The method according to any one of claims 1-6, wherein the subject is administered at least 900 mg of a compound of Formula (1) per day.

10. The method according to any one of claims 1-6, wherein the subject is administered at least 1200 mg of a compound of Formula (1) per day.

11. The method according to any one of claims 1-6, wherein the subject is administered a daily dosage between 300 to 1500 mg, 600 to 1250 mg, or 900 to 1200 mg of a compound of Formula (1) per day.

12. The method according to any one of claims 7-11, wherein the daily dosage is achieved by two administrations.

13. The method according to any one of claims 7-11, wherein the daily dosage is achieved by three administrations.

14. The method according to any one of claims 7-13, wherein the daily dosage is administered daily for at least six months.

15. The method according to any one of claims 7-13, wherein the daily dosage is administered daily for at least twelve months.

16. The method according to any one of claims 7-13, wherein the daily dosage is administered daily for at least eighteen months.

17. The method according to any one of claims 1-16, further comprising testing the subject for the presence of one or more markers in said subject before the treatment, wherein the markers are selected from the group consisting of amyloid plaques in the brain, neurofibrillary tangles in the brain, decline in brain glucose metabolism, decline in thiamine diphosphate-dependent enzyme activity in the blood or brain, and increase in advanced glycation end products in the blood, urine, or brain.

18. The method according to any one of claims 1-17, wherein the subject has been selected for treatment on the basis that the subject is positive for the presence of one or more markers selected from the group consisting of amyloid plaques in the brain, neurofibrillary tangles in the brain, decline in brain glucose metabolism, decline in thiamine diphosphate-dependent enzyme activity in the blood or brain, and increase in advanced glycation end products in the blood, urine, or brain.

19. The method according to any one of claims 1-18, further comprising monitoring the subject during the treatment to determine whether a decrease is observed in the level of one or more markers selected from the group consisting of amyloid plaques in the brain, neurofibrillary tangles in the brain, decline in brain glucose metabolism, decline in thiamine diphosphate-dependent enzyme activity in the blood or brain, and increase in advanced glycation end products in the blood, urine, or brain.

20. The method according to any one of claims 1-19, wherein the subject is not a carrier of apolipoprotein E4 (apoE4) allele.

21. The method according to any one of claims 1-20, further comprising testing the subject for apoE4 allele presence before the treatment.

22. The method according to any one of claims 1-21, wherein the subject has been selected for the treatment on the basis that the subject is not a carrier of apolipoprotein E4 (apoE4).

23. The method according to any one of claims 1-22, wherein R¹ is —R.

24. The method of claim 23, wherein R is an unsaturated hydrocarbon.

25. The method of claim 23, wherein R comprises phenyl.

26. The method of claim 23, wherein R is —C(O)R^{a}, wherein R^{a} is a hydrocarbon group containing 1-20 carbon atoms and optionally containing one or more heteroatoms selected from oxygen, nitrogen, and sulfur.

27. The method of claim 26, wherein R^{a} is an unsaturated hydrocarbon.

28. The method of claim 27, wherein R^{a} comprises phenyl.

29. The method according to any one of claims 1-28, wherein R² is —OPO₃²⁻.

30. The method according to any one of claims 1-28, wherein R² is —OR′ or —OC(O)R′.

31. The method according to any one of claims 1-22, wherein the compound according to Formula (1) is benfotiamine, or a pharmaceutically acceptable salt, solvate, or polymorph thereof.

32. A method for treating a subject, the method comprising:
administering to a subject who has been identified as positive for at least one marker, a pharmaceutically effective amount of a compound within the following generic structure: 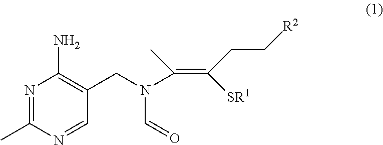
wherein R¹ is either —R or —SR, wherein R is a hydrocarbon group containing 1-20 carbon atoms and optionally containing one or more heteroatoms selected from oxygen, nitrogen, and sulfur; and R² is selected from the group consisting of —OR′, —OPO₃²⁻, and —OC(O)R′, wherein R′ is a hydrogen atom or a hydrocarbon group containing 1-6 carbon atoms;
wherein Formula (1) further comprises pharmaceutically acceptable salts, solvates, and polymorphs thereof, and
wherein the marker is selected from the group consisting of amyloid plaques in the brain, neurofibrillary tangles in the brain, decline in brain glucose metabolism, decline in thiamine diphosphate-dependent enzyme activity in the blood or brain, and increase in advanced glycation end products in the blood, urine, or brain.

33. The method of claim 32, wherein the subject does not yet display cognitive impairment at the start of treatment.

34. The method according to any one of claims 32 and 33, further comprising monitoring the level of the at least one marker in the subject.

35. The method according to any one of claims 32-34, wherein the administration of the compound is adjusted based on an observed change in the level of the at least one marker.

36. The method according to claim 35, wherein the adjustment comprises terminating the administration when the level of the at least one marker is diminished.

37. A method of increasing a peripheral blood level of thiamine in a subject having a decreased tissue level of thiamine, or decreased peripheral blood level of thiamine, wherein the decreased tissue level of thiamine or peripheral blood level of thiamine is not due to a dietary insufficiency of thiamine, comprising administering to the subject a pharmaceutically effective amount of a compound within the following generic structure: 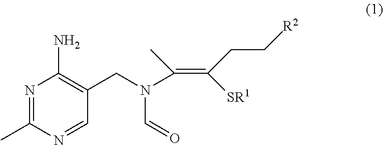
wherein R¹ is either —R or —SR, wherein R is a hydrocarbon group containing 1-20 carbon atoms and optionally containing one or more heteroatoms selected from oxygen, nitrogen, and sulfur; and R² is independently selected from the group consisting of —OR′, —OPO₃²⁻, and —OC(O)R′, wherein R′ is a hydrogen atom or a hydrocarbon group containing 1-6 carbon atoms;
or a pharmaceutically acceptable salt, solvate, or polymorph thereof,
wherein the pharmaceutically effective amount is sufficient to increase a tissue level or peripheral blood level of thiamine in the subject relative to the tissue level or peripheral blood level of thiamine, respectively, prior to treatment.

38. The method of claim 37, wherein administering to the subject a pharmaceutically effective amount of the compound also increases a tissue level or peripheral blood level of thiamine diphosphate and of thiamine monophosphate in the subject.

39. The method of claim 37 or 38, wherein the compound is administered daily for a period of over six months and wherein the peripheral blood level of thiamine in the subject at the end of treatment is increased at least 100-fold relative to the tissue level or peripheral blood level of thiamine prior to treatment.

40. The method of any of claims 37 to 39, wherein the method effects an increase in the tissue level or thiamine in the blood.
